(19) United States
(12) Patent Application Publication
(10) Pub. No.: US 2024/0011086 A1
(43) Pub. Date: Jan. 11, 2024

(54) ELECTRONIC DETECTION OF NUCLEIC ACID STRUCTURE

(71) Applicant: OMNIOME, INC., San Diego, CA (US)

(72) Inventors: Maxim ABASHIN, San Diego, CA (US); Pinar IYIDOGAN, San Diego, CA (US); John T. MURPHY, San Diego, CA (US); Alex NEMIROSKI, San Diego, CA (US); Min-Jui Richard SHEN, San Diego, CA (US); Michele Elizabeth WISNIEWSKI, San Diego, CA (US)

(73) Assignee: OMNIOME, INC., San Diego, CA (US)

(21) Appl. No.: 17/293,412

(22) PCT Filed: Sep. 10, 2019
(86) PCT No.: PCT/US2019/050443

§ 371 Date: Sep. 8, 2022

Related U.S. Application Data
(60) Provisional application No. 62/767,712, filed on Nov. 15, 2018.

(51) Int. Cl. C12Q 1/6869; G01N 27/414

(52) U.S. Cl. CPC C12Q 1/6869; G01N 27/4145

(57) ABSTRACT

A sensor having a first electrode and a second electrode operably connected by a conduction channel, wherein a polymerase, primed template nucleic acid and nucleotide form a stabilized ternary complex that is immobilized in or on the conduction channel, whereby association and dissociation of the ternary complex is detected due to changes in electrical properties of the sensor. Identification of the nucleotide type that participates in the complex indicates the identity of the next template base in the template. Repeated cycles of extending the primer and detecting stabilized ternary complexes can allow determination of the sequence of nucleotides for the template.

### CROSS REFERENCE TO RELATE APPLICATION

This application is based on, and claims the benefit of, U.S. Provisional Application No. 62/767,712, filed Nov. 15, 2018, which is incorporated herein by reference.

### BACKGROUND

The present disclosure relates generally to analytical detection of molecular biological events and has specific applicability to nucleic acid sequencing.

Nucleic acid sequencing technologies are quickly moving from scientific novelty to diagnostic revolution. Modern sequencing technologies, referred to as next generation sequencing (NGS) technologies, have become a routine tool for biological research. This has fueled discovery of genetic markers for a wide variety of diseases and conditions. The rate at which new markers are identified for diagnosis and prognosis continues to increase as commercial sequencing platforms become more accessible to biological researchers. However, the rate at which the discovered markers are being translated into the clinic, where the lives of patients can be impactfully benefitted, is still slow. Even in cases where clinical tests have been developed, the costs for performing the tests can be prohibitive except for the wealthiest and/or most at-risk patients.

The sequencing platforms that are currently in commercial use rely on a variety of detection technologies. Generally, the platforms facilitate a biochemical reaction that uses uniquely labeled molecular components to detect the sequence of nucleotides that is present in DNA. The platforms also include a detector component that distinguishes the labels. The most prominent platforms use optical detectors to distinguish fluorescently labeled nucleotides or fluorescently labeled oligonucleotides as they interact with the DNA that is to be sequenced. Although optical detection has found wide use, current platforms are limited by certain inherent disadvantages of optical detection. For example, high energy light is used by the detection systems and this leads to photodegradation of the very reaction components that are to be detected. This in turn reduces accuracy and throughput of optical platforms. Moreover, expensive optical components have been required in order to achieve the current state of accuracy and throughput. The costs are an impediment to clinical uptake.

Electrical detection is used in some commercial sequencing platforms. For example, nanopore sequencing detects electrical signals that result from passage of DNA or other reaction components through a nanopore. In other platforms, the DNA is subjected to biochemical reactions that release protons or other charged reaction components that can be detected to determine the nucleotide sequence of the DNA. Although electronic detection platforms avoid problems with photodegradation of DNA, the currently available commercial platforms that utilize electronic detection are less accurate and, in many cases, more expensive than optical-based platforms. High cost and low accuracy are an impediment to clinical uptake of platforms that use electrical detection.

Thus, there exists a need for sequencing platforms that utilize detection chemistries and detection hardware having accuracy and cost that will translate sequencing into clinical settings. The present invention satisfies this need and provides related advantages as well.

### BRIEF SUMMARY

The present disclosure provides a sensor that includes a first electrode, a second electrode, a conduction channel operably connecting the first electrode to the second electrode and a primed template nucleic acid that is immobilized at the sensor, wherein the primed template nucleic acid is bound to a polymerase and next correct nucleotide in a stabilized ternary complex, wherein the stabilized ternary complex is prevented from covalently incorporating the next correct nucleotide into the primed template nucleic acid.

The present disclosure further provides a sensor that includes a first electrode, a second electrode, a conduction channel operably connecting the first electrode to the second electrode and a polymerase that is immobilized at the sensor, wherein the polymerase is bound to a primed template nucleic acid and next correct nucleotide in a stabilized ternary complex, wherein the stabilized ternary complex is prevented from covalently incorporating the next correct nucleotide into the primed template nucleic acid.

Also provided by the present disclosure is a sensor that includes a first electrode that is attached to a first reaction component via a first conductive linker and a second electrode that is attached to a second reaction component via a second conductive linker, wherein formation of binding complex between the first reaction component and the second reaction component creates a conduction channel operably connecting the first electrode to the second electrode.

The present disclosure provides a method for identifying the next correct nucleotide for a primed template nucleic acid. The method can include steps of (a) providing a sensor that includes a first electrode, a second electrode, a conduction channel operably connecting the first electrode to the second electrode and a primed template nucleic acid that is immobilized at the sensor; (b) contacting the primed template nucleic acid with a polymerase and the next correct nucleotide, thereby forming a stabilized ternary complex that includes the primed template nucleic acid, the polymerase and the next correct nucleotide, wherein the stabilized ternary complex is prevented from covalently incorporating the next correct nucleotide into the primed template nucleic acid; and (c) monitoring the sensor to detect a signal produced by the stabilized ternary complex, whereby the next correct nucleotide is identified from the signal. Optionally, the above steps can be performed in a method for sequencing the primed template nucleic acid, wherein the method further includes steps of (d) adding a next correct nucleotide to the primer of the primed template nucleic acid after step (b), thereby producing an extended primer; and (e) repeating steps (a) through (d) for the primed template nucleic acid that includes the extended primer.

In an alternative configuration, a method for identifying the next correct nucleotide for a primed template nucleic acid can include steps of (a) providing a sensor that includes a first electrode, a second electrode, a conduction channel operably connecting the first electrode to the second electrode and a polymerase that is immobilized at the sensor; (b) contacting the polymerase with a primed template nucleic acid and the next correct nucleotide, thereby forming a stabilized ternary complex that includes the primed template nucleic acid, the polymerase and the next correct nucleotide, wherein the stabilized ternary complex is prevented from covalently incorporating the next correct nucleotide into the primed template nucleic acid; and (c) monitoring the sensor to detect a signal produced by the stabilized ternary complex, whereby the next correct nucleotide is identified from the signal. Optionally, the above steps can be performed in a method for sequencing the primed template nucleic acid, wherein the method further includes steps of (d) adding a next correct nucleotide to the primer of the primed template nucleic acid after step (b), thereby producing an extended primer; and (e) repeating steps (a) through (d) for the primed template nucleic acid that includes the extended primer.

A method for identifying the next correct nucleotide for a primed template nucleic acid can also include steps of (a) providing a sensor having a first electrode that is attached to a polymerase via a first conductive linker and a second electrode that is attached to a primed template nucleic acid via a second conductive linker; (b) contacting the polymerase and the primed template nucleic acid with the next correct nucleotide, thereby forming a stabilized ternary complex including the primed template nucleic acid, the polymerase and the next correct nucleotide, wherein the stabilized ternary complex is prevented from covalently incorporating the next correct nucleotide into the primed template nucleic acid; and (c) monitoring the sensor to detect a circuit between the first and second electrode produced by the stabilized ternary complex, whereby the next correct nucleotide is identified from the signal. Optionally, the above steps can be performed in a method for sequencing the primed template nucleic acid, wherein the method further includes steps of (d) adding a next correct nucleotide to the primer of the primed template nucleic acid after step (b), thereby producing an extended primer; and (e) repeating steps (a) through (d) for the primed template nucleic acid that includes the extended primer.

In other embodiments, a method for identifying the next correct nucleotide for a primed template nucleic acid can include steps of (a) providing a sensor having a first electrode that is attached to a polymerase via a first conductive linker and a second electrode that is attached to a nucleotide via a second conductive linker; (b) contacting the polymerase and the nucleotide with a primed template nucleic acid, thereby forming a stabilized ternary complex including the primed template nucleic acid, the polymerase and the nucleotide, wherein the stabilized ternary complex is prevented from covalently incorporating the nucleotide into the primed template nucleic acid; and (c) monitoring the sensor to detect a circuit between the first and second electrode produced by the stabilized ternary complex, whereby the nucleotide is identified as the next correct nucleotide for the primed template nucleic acid. Optionally, the above steps can be performed in a method for sequencing the primed template nucleic acid, wherein the method further includes steps of (d) adding a next correct nucleotide to the primer of the primed template nucleic acid after step (b), thereby producing an extended primer; and (e) repeating steps (a) through (d) for the primed template nucleic acid that includes the extended primer.

Alternatively, a method for identifying the next correct nucleotide for a primed template nucleic acid can include steps of (a) providing a sensor having a first electrode that is attached to a primed template nucleic acid via a first conductive linker and a second electrode that is attached to a nucleotide via a second conductive linker; (b) contacting the primed template nucleic acid and the nucleotide with a polymerase, thereby forming a stabilized ternary complex including the primed template nucleic acid, the polymerase and the nucleotide, wherein the stabilized ternary complex is prevented from covalently incorporating the nucleotide into the primed template nucleic acid; and (c) monitoring the sensor to detect a circuit between the first and second electrode produced by the stabilized ternary complex, whereby the nucleotide is identified as the next correct nucleotide for the primed template nucleic acid. Optionally, the above steps can be performed in a method for sequencing the primed template nucleic acid, wherein the method further includes steps of (d) adding a next correct nucleotide to the primer of the primed template nucleic acid after step (b), thereby producing an extended primer; and (e) repeating steps (a) through (d) for the primed template nucleic acid that includes the extended primer.

### BRIEF DESCRIPTION OF THE DRAWINGS

FIG. 1 shows an electronic sensor having a nucleic acid linked to a conduction channel and formation of ternary complexes on the nucleic acid using various combinations of labeled and non-labelled binding components.

FIG. 2 shows an electronic sensor having a polymerase linked to a conduction channel and formation of ternary complexes on the polymerase using various combinations of labeled and non-labelled binding components.

FIG. 3 shows an electronic sensor having a nucleic acid that connects two electrodes to form a circuit and formation of ternary complexes on the nucleic acid using various combinations of labeled and non-labelled binding components.

FIG. 4 shows an electronic sensor having a polymerase that connects two electrodes to form a circuit and formation of ternary complexes on the polymerase using various combinations of labeled and non-labelled binding components.

FIG. 5 shows an electronic sensor having a nucleic acid linked to one electrode and a polymerase linked to a second electrode, wherein the linkers are sufficiently flexible and long to allow the polymerase and nucleic acid to form a stabilized ternary complex with a nucleotide, and wherein the ternary complex completes a circuit with the two electrodes.

FIG. 6 shows an electronic sensor having a nucleic acid linked to one electrode and different nucleotide types linked to four other electrodes, respectively, wherein the linkers are sufficiently flexible and long to allow the nucleic acid and one of the nucleotides to form a stabilized ternary complex with a polymerase, and wherein the ternary complex completes a circuit with the electrodes for the nucleic acid and bound nucleotide.

FIG. 7 shows an electronic sensor having a polymerase linked to one electrode and different nucleotide types linked to four other electrodes, respectively, wherein the linkers are sufficiently flexible and long to allow the polymerase and one of the nucleotides to form a stabilized ternary complex with a nucleic acid, and wherein the ternary complex completes a circuit with the electrodes for the polymerase and bound nucleotide.

### DETAILED DESCRIPTION

The present disclosure provides electronic sensors configured to detect presence or absence of a ternary complex that contains a polymerase, primed template nucleic acid and nucleotide. Detection of the presence or absence of ternary complexes can be used to identify the base composition of the template nucleic acid. For example, the base that is present at a particular position of a template can be determined from knowledge of the type of nucleotide that is present in the ternary complex, because complementarity between the nucleotide and template base follows predictable rules (i.e. Watson-Crick base pairing rules). The type of nucleotide that is present in a ternary complex can be determined using a variety of techniques and sensor configurations set forth herein. Repeated cycles of extending the primer and detecting stabilized ternary complexes can allow determination of the sequence of bases in the template.

Use of electronic sensors as set forth herein provides several advantages over commercially available sequencing platforms. Several sensor configurations set forth herein provide single molecule detection such as sequencing of single nucleic acid molecules. In contrast, several commercially available sequencing platforms require that nucleic acid molecules be amplified to form amplicon clusters on a surface or bead prior to sequencing. This preliminary amplification step is prone to introduce errors into the template, which translates into errors in the sequencing data. The single molecule methods of the present disclosure avoid errors introduced during amplicon cluster formation and can thus provide the advantage of more accurate sequencing results.

Electronic sensors of the present disclosure also provide an advantage of reducing noise from background signals. Several commercially available sequencing platforms rely on optical detection of fluorescently labeled reaction components during sequencing reactions. Generally, signal can be acquired from nucleic acids as the labeled components are recruited to the nucleic acids in the course of sequencing. However, background fluorescence arising from labeled components in the environment around the nucleic acids typically produce background signals that adversely impact signal to noise, specificity of detection and, ultimately, sequencing accuracy. In contrast, electronic sensors provided herein detect ternary complex association and dissociation based on detection of reaction components that are proximal to the sensor. As such, the majority of background components diffuse in solution and are not detected by the sensor. Proximity detection using a sensor of the present disclosure is particularly advantageous when detecting equilibrium binding reactions since unbound reactants and bound products are present together under equilibrium detection. By way of more specific example, proximity-based detection provides reduced background when detecting presence of a ternary complex in the presence of unbound components of the complex (i.e. unbound primed template nucleic acid, unbound polymerase and unbound nucleotide), compared to techniques that detect optically labeled component in solution or on a surface in a flow cell.

A further advantage of electronic detection using a sensor and/or method set forth herein is avoidance of photodamage. Several currently available sequencing platforms rely on fluorescent detection. These platforms blast nucleic acids and other reaction components with high intensity excitation light, typically from a laser. The resulting photodamage has an adverse impact on the length of reads that can be acquired before the DNA being sequenced becomes compromised beyond use and also adversely impact accuracy of base calling.

Sequencing By Binding™ (SBB™) techniques provide a previously unrecognized advantage as a reaction format for sequencing nucleic acids on an electronic sensor, especially in comparison to sequencing by synthesis techniques. Sequencing By Synthesis can be detected using a field effect transistor (FET) that is configured to detect subtle conformational changes that occur as a nucleotide is incorporated into a primer by a polymerase. However, the changes are subtle and difficult to distinguish. Also, the conformational changes are typically observed in the time domain which requires data acquisition over a substantial period of time. This results in financial and time costs associated with acquiring the data, processing signals and storing data. In contrast, SBB™ results in far less subtle changes at the sensor due to formation of a ternary complex. Also, the presence or absence of ternary complex can be detected as an end point reading without requiring time-based signal acquisition. Thus, SBB™ can provide a more accurate and robust sequencing technique for electronic detection compared to Sequencing By Synthesis (SBS) techniques.

### Definitions

Terms used herein will be understood to take on their ordinary meaning in the relevant art unless specified otherwise. Several terms used herein and their meanings are set forth below.

As used herein, the term “ambiguous,” when used in reference to a signal, means that the signal apparently has more than one potential origin. For example, an ambiguous signal that is acquired in a cycle of a sequencing reaction may not distinguish between two or more nucleotide types that could participate in the cycle to produce the signal. When used in reference to a nucleic acid representation (e.g. a nucleic acid sequence), the term “ambiguous” refers to a position in the nucleic acid representation for which two or more nucleotide types are identified as candidate occupants. An ambiguous position can have, for example, at least 2, 3 or 4 nucleotide types as candidate occupants. Alternatively or additionally, an ambiguous position can have at most 4, 3 or 2 nucleotide types as candidate occupants.

As used herein, the term “attached” refers to the state of two things being joined, fastened, adhered, connected or bound to each other. For example, a reaction component, such as a primed template nucleic acid or a polymerase, can be attached to a solid phase component, such as a charge sensor, by a covalent or non-covalent bond. A covalent bond is characterized by the sharing of pairs of electrons between atoms. A non-covalent bond is a chemical bond that does not involve the sharing of pairs of electrons and can include, for example, hydrogen bonds, ionic bonds, van der Waals forces, hydrophilic interactions and hydrophobic interactions.

As used herein, the term “binary complex” refers to an intermolecular association between a polymerase and a primed template nucleic acid, lacking a monomeric nucleotide molecule such as a next correct nucleotide for the primed template nucleic acid.

As used herein, the term “blocking moiety,” when used in reference to a nucleotide, means a part of the nucleotide that inhibits or prevents the 3′ oxygen of the nucleotide from forming a covalent linkage to the next correct nucleotide during a nucleic acid polymerization reaction. The blocking moiety of a “reversibly terminated” nucleotide can be removed from the nucleotide analog, or otherwise modified, to allow the 3′-oxygen of the nucleotide to covalently link to a next correct nucleotide. Such a blocking moiety is referred to herein as a “reversible terminator moiety.” Exemplary reversible terminator moieties are set forth in U.S. Pat. Nos. 7,427,673; 7,414,116; 7,057,026; 7,544,794 or 8,034,923; or PCT publications WO 91/06678 or WO 07/123744, each of which is incorporated herein by reference. A nucleotide that has a blocking moiety or reversible terminator moiety can be at the 3′ end of a nucleic acid, such as a primer, or can be a monomer that is not covalently attached to a nucleic acid. A particularly useful blocking moiety will be present at the 3′ end of a nucleic acid that participates in formation of a ternary complex.

As used herein, the term “catalytic metal ion” refers to a metal ion that facilitates phosphodiester bond formation between the 3′-oxygen of a nucleic acid (e.g., a primer) and the phosphate of an incoming nucleotide by a polymerase. A “divalent catalytic metal cation” is a catalytic metal ion having a valence of two. Catalytic metal ions can be present at concentrations that stabilize formation of a complex between a polymerase, nucleotide, and primed template nucleic acid, referred to as non-catalytic concentrations of a metal ion insofar as phosphodiester bond formation does not occur. Catalytic concentrations of a metal ion refer to the amount of a metal ion sufficient for polymerases to catalyze the reaction between the 3′-oxygen of a nucleic acid (e.g., a primer) and the phosphate group of an incoming nucleotide.

As used herein, the term “code,” means a system of rules to convert information, such as signals obtained from a detection apparatus, into another form or representation, such as a base call or nucleic acid sequence. For example, signals that are produced by one or more ternary complex having a particular type of bound nucleotide can be encoded by a digit. The digit can have several potential values, each value encoding a different signal state. For example, a binary digit will have a first value for a first signal state and a second value for a second signal state. A digit can have a higher radix including, for example, a ternary digit having three potential values, a quaternary digit having four potential values etc. A series of digits can form a codeword. For example, the series of digits can encode a series of signal states acquired from a series of ternary complex examination steps. The length of the codeword is the same as the number of examination steps performed. Exemplary codes include, but are not limited to, a repetition code, parity code, error detecting code, error correcting code, linear code or Hamming code.

The term “comprising” is intended herein to be open-ended, including not only the recited elements, but further encompassing any additional elements.

As used herein, the term “deblock” means to remove or modify a reversible terminator moiety of a nucleotide to render the nucleotide extendable. For example, the nucleotide can be present at the 3′ end of a primer such that deblocking renders the primer extendable. Exemplary deblocking reagents and methods are set forth in U.S. Pat. Nos. 7,427,673; 7,414,116; 7,057,026; 7,544,794 or 8,034,923; or PCT publications WO 91/06678 or WO 07/123744, each of which is incorporated herein by reference.

As used herein, the term “each,” when used in reference to a collection of items, is intended to identify an individual item in the collection but does not necessarily refer to every item in the collection. Exceptions can occur if explicit disclosure or context clearly dictates otherwise.

As used herein, the term “electrode” refers to a structure that can act as an efficient source or sink of charge carriers. Exemplary electrodes include metal or semiconductor structures, such as those used in electronic circuits. A pair of spaced apart electrodes can function as a source and drain electrode pair. In various configurations disclosed herein, a sensor may include a gate electrode. When present, a gate electrode is used to apply a voltage rather than transfer charge carriers. Thus, a gate electrode can support accumulation of charge carriers to produce a local electric field, but is not intended to pass current. In this configuration a gate electrode will be electrically isolated from the primary conduction paths of a circuit by some form of insulating layer or material.

As used herein, the term “error correcting code” means a code that provides recovery of valid information. For example, an error correcting code can have sufficient information to recover valid signals from invalid signals or to make a valid base call from invalid or erroneous signals. An error correcting code can function as an error detecting code.

As used herein, the term “error detecting code” means a code that identifies information as being valid or invalid. For example, an error detecting code can have sufficient information to distinguish valid signals from invalid signals or to distinguish a valid base call from an invalid base call.

As used herein, the term “exogenous,” when used in reference to a moiety of a molecule, means a chemical moiety that is not present in a natural analog of the molecule. For example, an exogenous label of a nucleotide is a label that is not present on a naturally occurring nucleotide. Similarly, an exogenous label that is present on a polymerase is not found on the polymerase in its native milieu.

As used herein, the term “extension,” when used in reference to a nucleic acid, means a process of adding at least one nucleotide to the 3′ end of the nucleic acid. The term “polymerase extension,” when used in reference to a nucleic acid, refers to a polymerase catalyzed process of adding at least one nucleotide to the 3′ end of the nucleic acid. A nucleotide or oligonucleotide that is added to a nucleic acid by extension is said to be incorporated into the nucleic acid. Accordingly, the term “incorporating” can be used to refer to the process of joining a nucleotide or oligonucleotide to the 3′ end of a nucleic acid by formation of a phosphodiester bond.

As used herein, the term “extendable,” when used in reference to a nucleotide, means that the nucleotide has an oxygen or hydroxyl moiety at the 3′ position, and is capable of forming a covalent linkage to a next correct nucleotide if and when incorporated into a nucleic acid. An extendable nucleotide can be at the 3′ position of a primer or it can be a monomeric nucleotide. A nucleotide that is extendable will lack blocking moieties such as reversible terminator moieties.

As used herein, the term “immobilized,” when used in reference to a molecule, refers to direct or indirect, covalent or non-covalent attachment of the molecule to a solid support. In some configurations, covalent attachment may be preferred, but generally all that is required is that the molecules (e.g. nucleic acids) remain immobilized or attached to the support under the conditions in which it is intended to use the support, for example, in applications that utilize immobilization of nucleic acid or polymerase at or near a sensor.

As used herein, the term “impute,” when used in reference to nucleotide identity, means inferring the presence of a particular type of nucleotide at a position in the nucleic acid absent observation of a detectable event attributable to the nucleotide. For example, the presence of a first nucleotide type at a position in a nucleic acid can be imputed based on absence of an observed signal for the first nucleotide type. Optionally, the imputation of the first nucleotide type's presence at the position can be further influenced by the observation of signal(s) for one or more other nucleotide type at the position.

As used herein, the term “label” refers to a molecule, or moiety thereof, that provides a detectable characteristic. The detectable characteristic can be, for example, an electrical property such as charge polarity (e.g. positive charge, negative charge or neutral charge for a label) or charge magnitude (e.g. number of elementary charges present in a label); optical property such as absorbance of radiation, fluorescence emission, luminescence emission, fluorescence lifetime, fluorescence polarization, or the like; Rayleigh and/or Mie scattering; binding affinity for a ligand or receptor; magnetic properties; mass; radioactivity or the like. Exemplary labels include, without limitation, a fluorophore, luminophore, chromophore, nanoparticle (e.g., gold, silver, carbon nanotubes), heavy atoms, radioactive isotope, mass label, magnetic label, charge label (e.g. positive charge label or negative charge label), spin label, receptor, ligand, or the like.

As used herein, “linker” and “chemical linker” and “crosslinker” all refer to a molecular construct that covalently joins two different entities. Exemplary entities that can be linked include, but are not limited to, a solid support, moiety or molecule (e.g., a plastic polymer and a plastic-adsorbent polymeric carrier protein) to each other.

As used herein, the term “next correct nucleotide” refers to the nucleotide type that will bind and/or incorporate at the 3′ end of a primer to complement a base in a template strand to which the primer is hybridized. The base in the template strand is referred to as the “next base” and is immediately 5′ of the base in the template that is hybridized to the 3′ end of the primer. The next correct nucleotide can be referred to as the “cognate” of the next base and vice versa. Cognate nucleotides that interact with each other in a ternary complex or in a double stranded nucleic acid are said to “pair” with each other. In accordance with Watson-Crick pairing rules adenine (A) pairs with thymine (T) or uracil (U), and cytosine (C) pairs with guanine (G). A nucleotide having a base that is not complementary to the next template base is referred to as an “incorrect”, “mismatch” or “non-cognate” nucleotide for the next template base.

As used herein, the term “non-catalytic metal ion” refers to a metal ion that, when in the presence of a polymerase enzyme, does not facilitate phosphodiester bond formation needed for chemical incorporation of a nucleotide into a primer. A non-catalytic metal ion may interact with a polymerase, for example, via competitive binding compared to catalytic metal ions. Accordingly, a non-catalytic metal ion can act as an inhibitory metal ion. A “divalent non-catalytic metal ion” is a non-catalytic metal ion having a valence of two. Examples of divalent non-catalytic metal ions include, but are not limited to, Ca²⁺, Zn²⁺, Co²⁺, Ni²⁺, and Sr²⁺. The trivalent Eu³⁺ and Tb³⁺ ions are non-catalytic metal ions having a valence of three.

As used herein, the term “nucleotide” can be used to refer to a native nucleotide or analog thereof. Examples include, but are not limited to, nucleotide triphosphates (NTPs) such as ribonucleotide triphosphates (rNTPs), deoxyribonucleotide triphosphates (dNTPs), or non-natural analogs thereof such as dideoxyribonucleotide triphosphates (ddNTPs) or reversibly terminated nucleotide triphosphates (rtNTPs).

As used herein, the term “polymerase” can be used to refer to a nucleic acid synthesizing enzyme, including but not limited to, DNA polymerase, RNA polymerase, reverse transcriptase, primase and transferase. Typically, the polymerase has one or more active sites at which nucleotide binding and/or catalysis of nucleotide polymerization may occur. The polymerase may catalyze the polymerization of nucleotides to the 3′ end of the first strand of the double stranded nucleic acid molecule. For example, a polymerase catalyzes the addition of a next correct nucleotide to the 3′ oxygen group of the first strand of the double stranded nucleic acid molecule via a phosphodiester bond, thereby covalently incorporating the nucleotide to the first strand of the double stranded nucleic acid molecule. Optionally, a polymerase need not be capable of nucleotide incorporation under one or more conditions used in a method set forth herein. For example, a mutant polymerase may be capable of forming a ternary complex but incapable of catalyzing nucleotide incorporation.

As used herein, the term “primed template nucleic acid” or “primed template” refers to a nucleic acid having a double stranded region such that one of the strands is a primer and the other strand is a template. The two strands can be parts of a contiguous nucleic acid molecule (e.g. a hairpin structure) or the two strands can be separable molecules that are not covalently attached to each other.

As used herein, the term “primer” refers to a nucleic acid having a sequence that binds to a nucleic acid at or near a template sequence. Generally, the primer binds in a configuration that allows replication of the template, for example, via polymerase extension of the primer. The primer can be a first portion of a nucleic acid molecule that binds to a second portion of the nucleic acid molecule, the first portion being a primer sequence and the second portion being a primer binding sequence (e.g. a hairpin primer). Alternatively, the primer can be a first nucleic acid molecule that binds to a second nucleic acid molecule having the template sequence. A primer can consist of DNA, RNA or analogs thereof. A primer can have an extendible 3′ end or a 3′ end that is blocked from primer extension.

As used herein, the term “signal” refers to energy or coded information that can be selectively observed over other energy or information such as background energy or background information. A signal can have a desired or predefined characteristic. For example, an electrical signal can be characterized or observed by one or more of intensity, polarity, current, voltage, impedance, resistance, conductance, capacity, electric field strength or the like. Absence of signal is understood to be a signal level of zero or a signal level that is not meaningfully distinguished from noise.

As used herein, the term “signal state” refers to a mode or characteristic of a signal obtained from a sensor or detector. Exemplary modes or characteristics include, but are not limited to, intensity, polarity, current, voltage, impedance, resistance, capacity, electric field strength etc. A signal state can have multiple potential values. For example, a signal state can have two potential states (binary), three potential states (ternary), four potential states (quaternary) etc.

As used herein, the term “ternary complex” refers to an intermolecular association between a polymerase, a double stranded nucleic acid and a nucleotide. Typically, the polymerase facilitates interaction between a next correct nucleotide and a template strand of the primed nucleic acid. A next correct nucleotide can interact with the template strand via Watson-Crick hydrogen bonding. The term “stabilized ternary complex” means a ternary complex having promoted or prolonged existence or a ternary complex for which disruption has been inhibited. Generally, stabilization of the ternary complex prevents covalent incorporation of the nucleotide component of the ternary complex into the primed nucleic acid component of the ternary complex.

As used herein, the term “type” is used to identify molecules that share the same chemical structure. For example, a mixture of nucleotides can include several dCTP molecules. The dCTP molecules will be understood to be the same type of nucleotide as each other, but a different type of nucleotide compared to dATP, dGTP, dTTP etc. Similarly, individual DNA molecules that have the same sequence of nucleotides are the same type, whereas DNA molecules with different sequences are different types. The term “type” can also identify moieties that share the same chemical structure. For example, the cytosine bases in a template nucleic acid will be understood to be the same type of base as each other independent of their position in the template sequence.

The embodiments set forth below and recited in the claims can be understood in view of the above definitions.

### Sensor Devices

The present disclosure provides electronic sensors for detecting molecular events such as binding reactions. The sensors can be configured to be sensitive enough to detect at the single molecule level. Detection of analytical reactions, such as formation of molecular complexes, can be accomplished using an electrically-conducting channel that has a single sensitizing molecule attached thereto or attached to a solid support surface that is proximate to the channel. Alternatively, detection of analytical reactions, such as association or dissociation of molecular complexes, can be accomplished using a first electrode having an electrically-conducting linker attached to a first complex-forming component and a second electrode having an electrically-conducting linker attached to a second complex-forming component, wherein formation of the complex can be detected as existence of a circuit between the two electrodes and dissociation of the complex can be detected as absence of the circuit. Accordingly, devices of the present disclosure can be used to detect or monitor the formation of a ternary complex that forms between a polymerase, primer template nucleic acid and next correct nucleotide, and can be used for the examination step of a single molecule sequencing reaction.

The present disclosure provides a sensor that includes a first electrode, a second electrode, a conduction channel operably connecting the first electrode to the second electrode and a primed template nucleic acid that is immobilized at the sensor, wherein the primed template nucleic acid is bound to a polymerase and next correct nucleotide in a stabilized ternary complex, wherein the stabilized ternary complex is prevented from covalently incorporating the next correct nucleotide into the primed template nucleic acid.

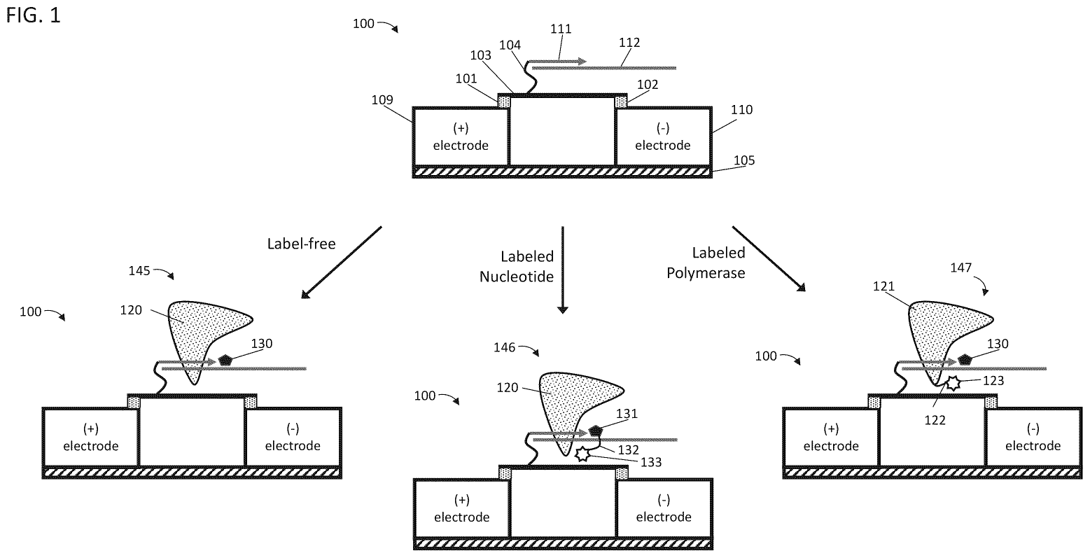

A primed template nucleic acid can be immobilized at a sensor via a linker that attaches the primed template nucleic acid to a conduction channel that operably connects two electrodes. An exemplary configuration is shown in FIG. 1. In this configuration, the sensor 100 includes a non-conducting support 105, a first electrode 109, a second electrode 110 and a conduction channel 103 that operably connects the first electrode 109 to the second electrode 110 to form a circuit. The conduction channel 103 connects to the first electrode 109 via attachment moiety 101 and to the second electrode 110 via attachment moiety 102. The sensor 100 includes at least one linker 104 attaching a nucleic acid to the channel. The linker is attached to the primer 111 in the figure, but the linker can be attached to template 112 instead of, or in addition to, being attached to the primer 111. Optionally, the linker 104 can have first and second functional groups, the first functional group of the linker 104 being attached to the conduction channel 103 and the second functional group of the linker being attached to the primer 111 or template 112. The linker 104 can be configured to conduct current or voltage from the nucleic acid to the conductive channel 103, or alternatively, the linker 104 can be insulated to prevent the flow of current or voltage.

As shown in FIG. 1, a ternary complex can be formed and detected using sensor 100. In the first configuration shown in FIG. 1, the sensor 100 is contacted with a polymerase 120 and next correct nucleotide 130 under conditions that promote formation of a stabilized ternary complex 145 that includes the polymerase 120, primer 111/template 112 hybrid, and next correct nucleotide 130. In this first configuration, presence of the ternary complex 145 can be detected based on a first signal state produced by the sensor 100 when it is present. Conversely, dissociation of the ternary complex can be observed as a second signal state produced by the sensor. Labels need not be used since the native charges on the polymerase can be sensed. In a second configuration, the sensor 100 is contacted with a polymerase 120 and a next correct nucleotide 131 that is attached to a charge label 133 via a linker 132 under conditions that promote formation of a stabilized ternary complex 146 that includes the polymerase 120, primer 111/template 112 hybrid, and labeled next correct nucleotide 131. In the second configuration of FIG. 1, the presence of ternary complex 146 is detected based, at least in part, on a first signal state produced by the sensor 100 when the label 133 is present. Dissociation of the ternary complex can be detected due to a second signal state for the sensor. In the third configuration shown, the sensor 100 is contacted with a polymerase 121 and a next correct nucleotide 130 under conditions that promote formation of a stabilized ternary complex 147 that includes the labeled polymerase 121, primer 111/template 112 hybrid, and next correct nucleotide 130. Polymerase 121 is attached to a charge label 123 via a linker 122. In the third configuration of FIG. 1, the presence of ternary complex 147 is detected based, at least in part, on a first signal state produced by the sensor 100 when the label 123 is present. Dissociation of the ternary complex can be detected due to a second signal state for the sensor.

In the example of FIG. 1, the polymerase and nucleotide are in solution phase when delivered to sensor 100. If desired the polymerase or nucleotide can be immobilized at or near the sensor. Immobilization can provide for increased speed of ternary complex formation due to constrained proximity of the complex-forming components. Immobilization can also provide cost savings by eliminating a need to replace polymerase or nucleotide during repeated cycles of a sequencing reaction or other repetitive analytical process. A polymerase or nucleotide can be immobilized at or near sensor 100 via a linker that conducts current or voltage between the polymerase, or nucleotide, and the sensor. Alternatively, the linker can be configured to not conduct current or voltage.

The present disclosure further provides a sensor that includes a first electrode, a second electrode, a conduction channel operably connecting the first electrode to the second electrode and a polymerase that is immobilized at the sensor, wherein the polymerase is bound to a primed template nucleic acid and next correct nucleotide in a stabilized ternary complex, wherein the stabilized ternary complex is prevented from covalently incorporating the next correct nucleotide into the primed template nucleic acid.

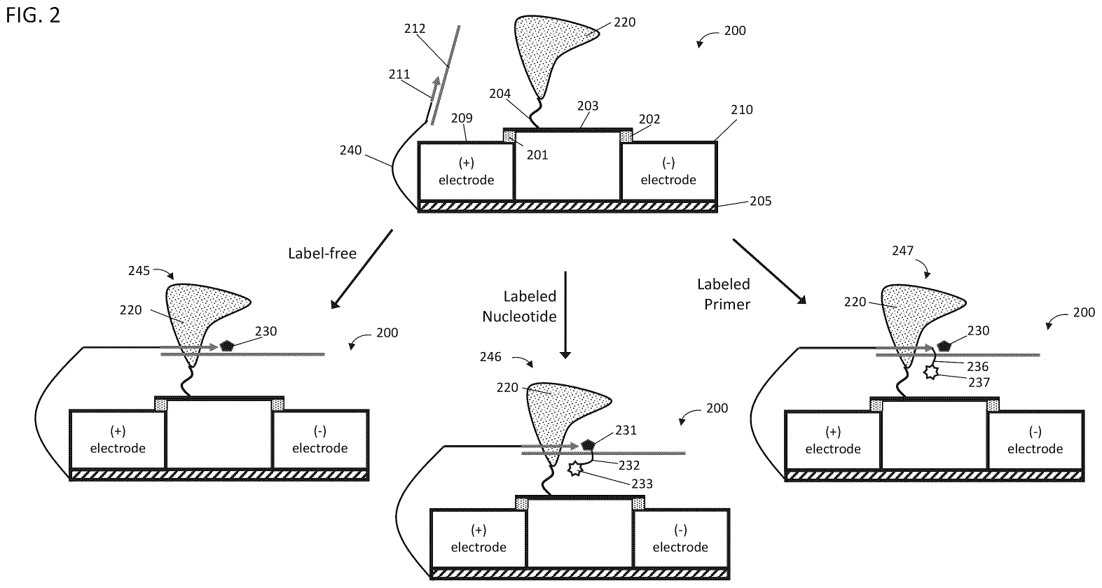

A polymerase can be immobilized at the sensor via a linker that attaches the polymerase to the conduction channel. An exemplary configuration is shown in FIG. 2. In this configuration, the sensor 200 includes a non-conducting support 205, a first electrode 209, a second electrode 210 and a conduction channel 203 that operably connects the first electrode 209 to the second electrode 210 to form a circuit. The conduction channel 203 connects to the first electrode 209 via attachment moiety 201 and to the second electrode 210 via attachment moiety 202. The sensor 200 includes at least one linker 204 attaching polymerase 220 to channel 203. Optionally, the linker 204 can have first and second functional groups, the first functional group of the linker 204 being attached to the conduction channel 203 and the second functional group of the linker 204 being attached to the polymerase 220. The linker 204 can be configured to conduct current or voltage from the polymerase to the conductive channel 103, or alternatively, the linker 104 can be insulated to prevent the flow of current or voltage. A primer 211/template 212 hybrid can optionally be immobilized to sensor 200 or to a solid support that is proximal to sensor 200. The linker 240 that immobilizes the primed template hybrid need not be conductive, for example being attached to non-conductive support 205. However, a conductive linker can be used to attach the nucleic acid to the sensor if desired. Furthermore, the nucleic acid need not be linked to the sensor and can instead be delivered to the sensor in solution phase.

As shown in FIG. 2, a ternary complex can be formed and detected using sensor 200. In the first configuration shown in FIG. 2, the sensor 200 is contacted with a next correct nucleotide 230 under conditions that promote formation of a stabilized ternary complex 245 that includes the polymerase 220 and primer 211/template 212 hybrid, and the next correct nucleotide 230. Here, linker 240 has flexibility and length that facilitates contact between polymerase 220 and the primer 211/template 212 hybrid. In this first configuration, presence of the ternary complex 245 can be detected based on a first signal state produced by the sensor 200. Absence of the ternary complex 245 can be observed from a second signal state produced by sensor 200. Labels need not be used since the native charges on the nucleic acid and nucleotide can be sensed. In a second configuration, the sensor 200 is contacted with a polymerase 220 and a next correct nucleotide 231 that is attached to a charge label 233 via a linker 232 under conditions that promote formation of a stabilized ternary complex 246 that includes the polymerase 220, primer 211/template 212 hybrid, and labeled next correct nucleotide 231. In the second configuration of FIG. 2, the presence of ternary complex 246 is detected based, at least in part, on a first signal state produced by the sensor 200 when the label 233 is present. Absence of the ternary complex 246 can be observed from a second signal state produced by sensor 200. In the third configuration shown, the sensor 200 is contacted with a polymerase 220 and a next correct nucleotide 230 under conditions that promote formation of a stabilized ternary complex 247 that includes the polymerase 221, primer 211/template 212 hybrid, and next correct nucleotide 230. Primer 211 is attached to a charge label 237 via a linker 236. The charge label 237 can be attached to the 3′ end of the primer and can optionally function as a reversible terminator moiety, as set forth in further detail herein below. In the third configuration of FIG. 2, the presence of ternary complex 247 is detected based, at least in part, on a first signal state produced by the sensor 200 when the label 237 is present. Absence of the ternary complex 247 can be observed from a second signal state produced by sensor 200.

As exemplified by the sensors shown in FIG. 1 and FIG. 2, a reaction component can be attached to a conduction channel via a linker and, as such, the reaction component need not provide an essential conduction path between the electrodes. In alternative embodiments, a reaction component (e.g. a polymerase or nucleic acid) can be wired to electrodes in order to provide an essential conduction path between electrodes. In this way, the reaction component can form a circuit with the electrodes.

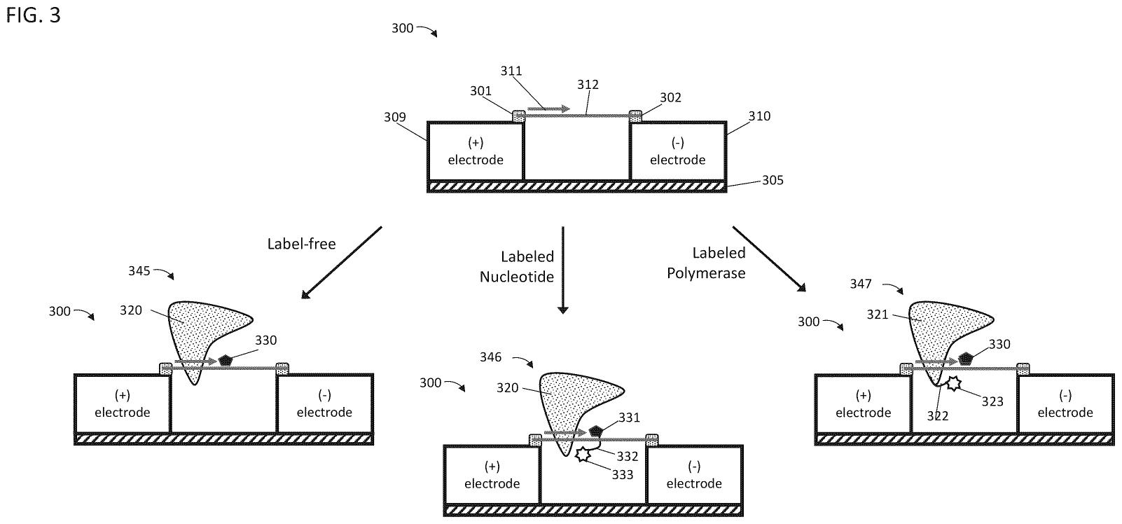

FIG. 3 shows exemplary sensor 300, which includes first electrode 309 and second electrode 310, the two electrodes being operably connected by template nucleic acid 312. The two electrodes are separated by a gap on non-conducting substrate 305 such that template nucleic acid 312 forms an essential conduction path between the electrodes. The template nucleic acid 312 is attached to electrode 309 via attachment moiety 301 and is attached to electrode 310 via attachment moiety 302.

As shown in FIG. 3, a ternary complex can be formed and detected using sensor 300. In the first configuration shown in FIG. 3, the sensor 300 is contacted with a polymerase 320 and next correct nucleotide 330 under conditions that promote formation of a stabilized ternary complex 345 that includes the polymerase 320, primer 311/template 312 hybrid, and next correct nucleotide 330. In this first configuration, presence or absence of the ternary complex 345 can be detected based on a first signal state produced by the sensor 300 when it is present and a second signal state when the complex is absent. Labels need not be used since the native charges on the polymerase and nucleotide can be sensed. In a second configuration, the sensor 300 is contacted with a polymerase 320 and a next correct nucleotide 331 that is attached to a charge label 333 via a linker 332 under conditions that promote formation of a stabilized ternary complex 346 that includes the polymerase 320, primer 311/template 312 hybrid, and labeled next correct nucleotide 331. In the second configuration of FIG. 3, the presence or absence of ternary complex 346 is detected based, at least in part, on a first signal state produced by the sensor 300 when the label 333 is present and a second signal state when the label is absent. In the third configuration shown, the sensor 300 is contacted with a polymerase 321 and a next correct nucleotide 330 under conditions that promote formation of a stabilized ternary complex 347 that includes the labeled polymerase 321, primer 311/template 312 hybrid, and next correct nucleotide 330. Polymerase 321 is attached to a charge label 323 via a linker 322. In the third configuration of FIG. 3, the presence or absence of ternary complex 347 is detected based, at least in part, on a first signal state produced by the sensor 300 when the label 323 is present and a second signal state when the label is absent.

In the example of FIG. 3, the polymerase and nucleotide are in solution phase when delivered to sensor 300. If desired the polymerase or nucleotide can be immobilized to sensor 300. Immobilization can provide for increased speed of ternary complex formation due to localized proximity of the complex-forming components. Immobilization can also provide cost savings by eliminating a need to replace polymerase or nucleotide during repeated cycles of a sequencing reaction or other repetitive analytical process. A polymerase or nucleotide can be immobilized to sensor 300 via a linker that conducts current or voltage between the polymerase, or nucleotide, and the sensor or the linker can be configured to not conduct current or voltage.

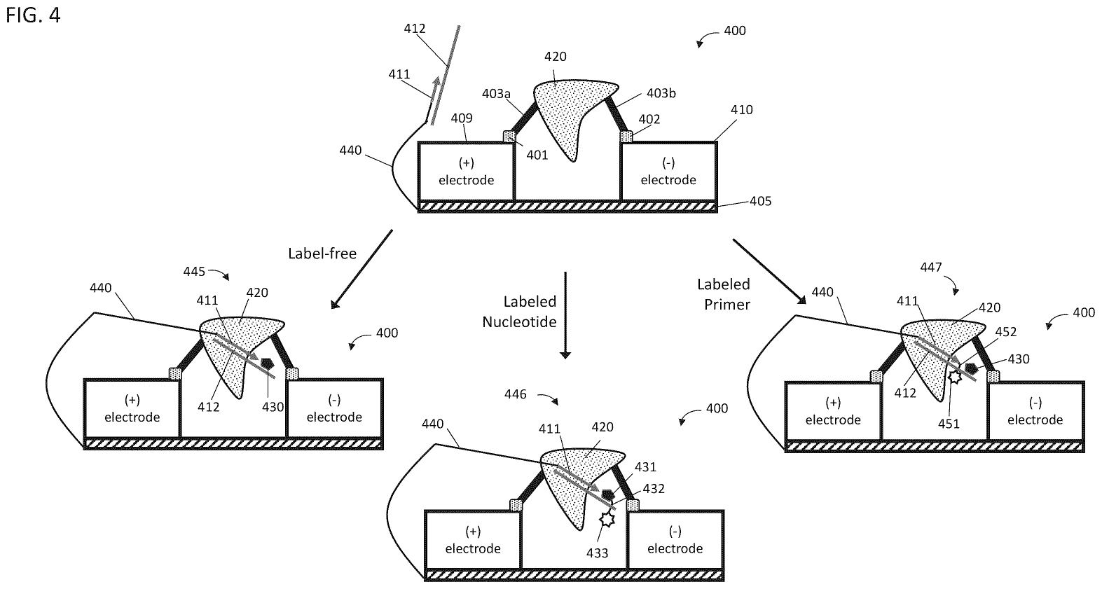

FIG. 4 shows exemplary sensor 400, which includes first electrode 409 and second electrode 410, the two electrodes being operably connected via polymerase 420. The two electrodes are separated by a gap on non-conducting substrate 405 such that polymerase 420 forms an essential conduction path between the electrodes. The polymerase 420 is attached to electrode 409 via linker 403a which is in turn attached to attachment moiety 401. The polymerase 420 is attached to electrode 410 via linker 403b which is in turn attached to attachment moiety 402. A primer 411/template 412 hybrid can optionally be immobilized to sensor 400 or to a solid support that is proximal to sensor 400. The linker 440 that immobilizes the primed template hybrid need not be conductive, for example being attached to non-conductive support 405. However, a conductive linker can be used to attach the nucleic acid to the sensor if desired. Furthermore, the nucleic acid need not be linked to the sensor and can instead be delivered to the sensor in solution phase.

As shown in FIG. 4, a ternary complex can be formed and detected using sensor 400. In the first configuration shown in FIG. 4, the sensor 400 is contacted with a next correct nucleotide 430 under conditions that promote formation of a stabilized ternary complex 445 that includes the polymerase 420, primer 411/template 412 hybrid, and next correct nucleotide 430. In this first configuration, presence or absence of the ternary complex 445 can be detected based on a first signal state produced by the sensor 400 when it is present and a second signal state of the sensor when the ternary complex is absent. Labels need not be used since the native charges on the primed template nucleic acid and nucleotide can be sensed. In a second configuration, the sensor 400 is contacted with a next correct nucleotide 431 that is attached to a charge label 433 via a linker 432 under conditions that promote formation of a stabilized ternary complex 446 that includes the polymerase 420, primer 411/template 412 hybrid, and labeled next correct nucleotide 431. In the second configuration of FIG. 4, the presence of ternary complex 446 is detected based, at least in part, on a first signal state produced by the sensor 400 when the label 433 is present and a second signal state of the sensor when the ternary complex is absent. In the third configuration shown, the sensor 400 is contacted with a next correct nucleotide 430 under conditions that promote formation of a stabilized ternary complex 447 that includes the polymerase 420, primer 411/template 412 hybrid, and next correct nucleotide 430. Primer 411 is attached to a charge label 451 via a linker 452. The charge label 451 can be attached to the 3′ end of primer 411 and can optionally function as a reversible terminator moiety, as set forth in further detail herein below. In the third configuration of FIG. 4, the presence of ternary complex 447 is detected based, at least in part, on a first signal state produced by the sensor 400 when the label 451 is present and a second signal state of the sensor when the ternary complex is absent.

Also provided by the present disclosure is a sensor that includes a first electrode that is attached to a first reaction component via a first conductive linker and a second electrode that is attached to a second reaction component via a second conductive linker, wherein formation of binding complex between the first reaction component and the second reaction component creates a conduction channel operably connecting the first electrode to the second electrode. For example, the first reaction component can be a polymerase and the second reaction component can be a primed template nucleic acid. As such, a binary complex can form between the polymerase and primed template nucleic acid to form a circuit. Alternatively, the polymerase and primed template nucleic acid can form a stabilized ternary complex with a next correct nucleotide. In an alternative configuration, one or more nucleotides can be attached to one of the electrodes instead of the polymerase or primed template nucleic acid. The component that is not immobilized can be provided in solution to form a stabilized ternary complex with the immobilized components.

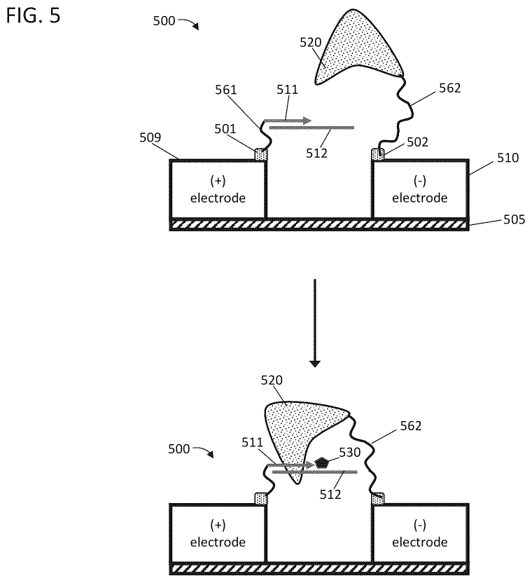

FIG. 5 shows sensor 500 in which non-conductive substrate 505 separates electrode 509 from electrode 510. Electrode 509 is attached to primer 511/template 512 hybrid via linker 561 and attachment moiety 501. Electrode 510 is attached to polymerase 520 via linker 562 and attachment moiety 502. The sensor can be contacted with nucleotide 530 such that a stabilized ternary complex forms between polymerase 520, primer 511/template 512 hybrid and nucleotide 530. Formation of the ternary complex 545 can be detected as the creation of a circuit between the electrodes because the attachment points 501 and 502, linkers 561 and 562, polymerase 520, and primer 511/template 512 hybrid are conductive. Dissociation of ternary complex can be detected as absence of a circuit between the electrodes.

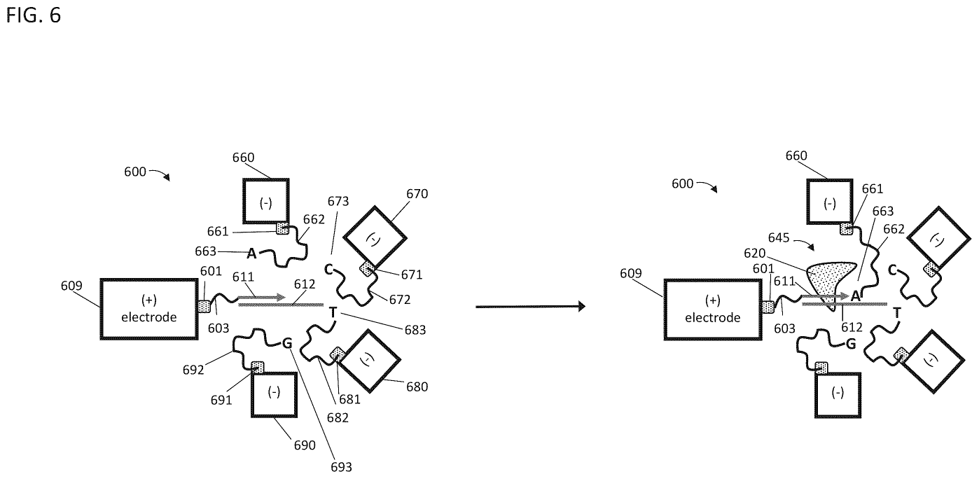

FIG. 6 shows sensor 600 in which non-conductive substrate 605 separates five electrodes from each other. Electrode 609 has positive polarity and is attached to primer 611/template 612 hybrid via linker 603 and attachment moiety 601. Electrodes 660, 670, 680 and 690 are attached to four different nucleotide types, respectively and have opposite polarity to the electrode 609 that is attached to the nucleic acid. Electrode 660 is attached to an Adenine nucleotide 663 via linker 662 and attachment moiety 661. The sensor can be contacted with polymerase 620 such that a stabilized ternary complex forms between polymerase 620, primer 611/template 612 hybrid and nucleotide 663 when thymine is the next template base. Formation of the stabilized ternary complex 645 can be detected as the creation of a circuit between the nucleic acid electrode 609 and the adenine electrode 660 because the attachment points 601 and 661, linkers 603 and 662, polymerase 620, and primer 611/template 612 hybrid are conductive. The identity of the next correct nucleotide for the stabilized complex can be determined based on the observation of a circuit formed with the adenine electrode 660 as opposed to the other nucleotide electrodes 670, 680 and 690. Dissociation of ternary complex can be detected as absence of a circuit between the electrodes.

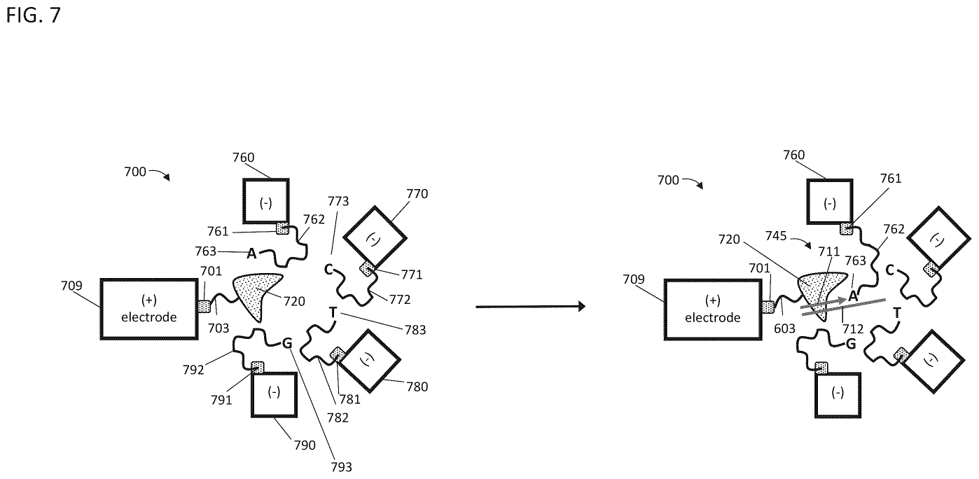

FIG. 7 shows sensor 700 which includes positive electrode 709 which is attached to polymerase 720 via conductive linker 703 and conductive attachment moiety 701. The polymerase electrode 709 is separated from four negative electrodes that are attached to four different nucleotide types, respectively. As is the case for sensor 600, the formation of a stabilized ternary complex can be detected based on creation of a circuit with one of the nucleotide electrodes. In the configuration shown, a circuit is formed between the polymerase electrode 709 and adenine electrode 760, thereby indicating that the next template base is thymine, the complement of the adenine 763 that is connected to electrode 760. Dissociation of ternary complex can be detected as absence of a circuit between the electrodes.

Any of a variety of sensors can be used in a method or apparatus of the present disclosure. Useful charge sensors include analytical devices that can accommodate a reaction component attached to, or in proximity to, a transduction element in a way that allows conversion of reaction events to detectable signals. In a standard field-effect transistor (FET), current flows along a conducting path (e.g. a conductive channel) that is connected to two electrodes, (e.g. a source and drain electrode). Exemplary sensors and methods for attaching a polymerase molecule to sensors via various linkers are set forth in US Pat. App. Pub. Nos. 2017/0240962 A1; 2018/0051316 A1; 2018/0112265 A1; 2018/0155773 A1 or 2018/0305727 A1; or U.S. Pat. Nos. 9,164,053; 9,829,456; each of which is incorporated herein by reference. Similar sensors and attachment techniques can be used for other reaction components such as nucleic acids or nucleotides

Any of a variety of different conduction channels that are generally found in field effect transistors can be used. Exemplary conduction channels are formed from metals, metal oxides, semiconductors, or nanometer-scale conductors such as nanowires, graphene, or single-walled carbon nanotubes (SWNTs). Other charge sensors that can be modified for use in an apparatus or method set forth herein include, for example, silicon nanowire (SiNW) FET, FET made of III-V materials, silicon FinFET, graphene nanoribbon FETs as well as nanoribbon FETs from other 2D materials such as MoS₂ and silicene, tunnel FET (TFET), and steep subthreshold slope devices (see, for example, Swaminathan et al., Proceedings of the 51st Annual Design Automation Conference on Design Automation Conference, pg. 1-6, ISBN: 978-1-4503-2730-5 (2014) and Ionescu et al., Nature 479, 329-337 (2011)).

A particularly useful channel for a charge sensor is a single-walled carbon nanotube (SWNT). A SWNT can be used for example, as a channel that operably connects electrodes and/or as a linker to attach a molecule that functions as a component of a reaction that is to be detected. As a class of materials, SWNTs are semiconductors with electronic bandgaps that can vary from 1 electron volt to effectively zero. In view of this variation carbon SWNTs can be classified as metallic or semi-metallic, and others as semiconducting. With the aid of connecting electrodes, electrostatic gates, and other control circuitry, semiconducting SWNTs can be configured as sensor FETs, as RF amplifiers, or as low-temperature single electron transistors. SWNTs are conduction channels in which single molecule sensing devices can be fabricated from SWNT wires of any type, with or without gate electrodes, and on glass, plastic, or silicon substrates. A sensing device described here can be one component within a FET or any number of more complex electronic or opto-electronic devices and circuitry. SWNTs and methods for their manufacture are set forth, for example, in Star et al., Nano. Lett. 3, 459 (2003); Star et al., Org. Lett. 6, 2089 (2004); Besterman et al., Nano. Lett. 3, 727 (2003); Gruner, Anal. Bioanal. Chem. 384, 322 (2005); Chen et al. Proc. Natl. Acad. Sci. U.S.A. 100, 4984 (2003) and US Pat App. Pub. No. 2013/0078622 A1, each of which is incorporated herein by reference.

A sensor can be coupled to electronic circuitry. The electronic circuitry can be used to apply a voltage bias (e.g., 50-100 mV) between a first electrode and a second electrode and can optionally be configured to measure the current flow as a function of time. Electronic circuitry may be coupled to a computer having one or more processors therein that is used to control the application of voltage and current through a sensor as well as to acquire, store, and/or analyze data generated by the sensor. During operation, a voltage (e.g., constant DC voltage or combination of AC and DC voltages) can be applied between the electrodes. The current then passing through the sensor can be measured using electronic circuitry, which may include a current meter with one or more amplifiers.

Various sensor components including, for example, a first electrode, second electrode, and conducting channel can be disposed on a substrate. The substrate can include any number of materials such as glass, quartz, sapphire, plastic, or silicon. The sensor can include, but does not require, a gate electrode or a conductive supporting substrate. Quartz is particularly useful for fabrication because it is compatible with high temperatures. Glass wafers can also be used if sensor components, such as SWNTs or other channels) are synthesized and deposited onto the substrate by certain processes, such as spin coating from solution, or if the devices are fabricated on wafers and then transferred to the glass for support.

Any of a variety of methods for measuring changes in electrical conductance can be used to monitor a sensor of the present disclosure. For example, a bias difference of 100 mV can be applied across a SWNT channel, and the current flowing through the conductor can be monitored using circuitry. Chemical binding or recognition on or near the sensor results in increases or decreases in the measured current. Multiple binding and dissociation events can produce multiple current fluctuations that can be timed, counted, discriminated, analyzed or stored using signal processing techniques which are known in the art.

An electronic sensor of the present disclosure may have its signal performance enhanced through various environmental factors. For example, the choice of pH buffer, salts and other additives, temperature and applied voltage may be modulated to improve the signal quality. In particular, the overall ionic strength of the buffer solution defines the Debye length in the solution, that is the distance over which the electric fields extend in solution. This can impact the extent to which current carriers passing through a reaction component are influenced by the charge distributions of components involved in the reaction and substrate. This can also impact the sensitivity of a FET sensor to a reaction component that is present in the electro-magnetic field of the sensor.

The driving voltage applied to a sensor can be optimized to improve the signaling from the sensor. Based on energy barriers within a reaction component that is linked to form a circuit with other sensor components, certain voltages may lead to improved signaling performance. In addition to an applied voltage, various configurations can also have a gate electrode, such as a buried gate within a substrate, such that voltages applied to the gate electrode further modulate the signaling properties of the sensor circuit. Certain embodiments may employ voltage spectroscopy, wherein the driving or gate voltages are swept through a range of values, and the signal of interest is in the response from this sweep, which contains information on the interaction between reaction components.

Electrodes and other electrically conductive components of a sensor set forth herein can optionally be covered. The cover may include a window, recess, slot, or other open segment that provides access from the external environment to the sensor. In this regard, the sensor can be exposed to a chemical environment. A protective covering protects the majority of the surface including the first and second electrodes from the environment. Moreover, in a preferred embodiment, the length of the window is tailored to achieve the correct device length. The length of the window can be varied to achieve the desired active region on the sensor. For example, first and second electrodes may be connected to a conducting channel (e.g. a SWNT channel) and separated by a distance of 2 μm. The window, however, can be made smaller than the inter-electrode distance. The exposed window within the protective covering exposes the conducting channel and the attached reaction component to the chemical environment. The protective cover can be any electrically-insulating film composed of one or more layers. Exemplary film materials include, but are not limited to, polymers, aluminum oxide, hafnium oxide, silicon dioxide, or silicon nitride. The window can be defined within the protective covering using lithographic techniques known in the art.

Optionally, device fabrication can include coating devices in a protective covering of positive electron beam resist such as polymethyl methacrylate (PMMA); writing lithographic patterns with an electron beam; and then developing the written areas to expose an active channel (e.g. SWNT channel having length in a range of 0.5 to 1.0 μm). In other fabrication techniques, devices can be coated with a protective covering of aluminum oxide; further coated in a film of optical photoresist; the desired windows exposed to light; the written areas developed to expose narrow windows of the aluminum oxide; the aluminum oxide etched to further expose the underlying channels (e.g. SWNT channel having length in a range of 0.5 to 1.0 μm). Combinations of two or more layers of materials in the protective coating can impart different chemical properties to the coated device.

Two or more electrodes that make up a sensor can be made from the same material as each other or, alternatively, different electrodes can have different compositions. For example, two or more electrodes can be made of metal. The metal can be the same for the two or more electrodes. Alternatively, a first electrode can be made from a first metal and a second electrode can be made from a different metal than the first electrode. Similarly, the attachment moiety on a first electrode can be the same material or different material compared to the attachment material on a second electrode. The use of different materials for electrodes or for their attachment moieties can provide a convenient means for attaching different molecular components to different electrodes.

### Immobilization of Reaction Components

In a particularly useful configuration a reaction component can be attached to a sensor or to a solid support surface that positions the reaction component in proximity to the sensor. For example, a component of a binding reaction such as a polymerase, nucleic acid (e.g. a primer and/or template), or nucleotide can be attached at or near a sensor. A molecule that functions as a reaction component can be immobilized in the field of the sensor via an electrically conductive linker. As such, current or voltage can flow between the sensor and the reaction component. For example, the reaction component can be immobilized to the sensor in a configuration that allows current or voltage to flow through the reaction component when current or voltage occurs between a source electrode and a drain electrode. Alternatively, a reaction component can be attached at or near a sensor using an insulated linker or insulated attachment that does not conduct voltage or current. In particular configurations, a single molecule of a particular reaction component is attached to a sensor. However, if desired an ensemble of similar molecules can be attached to a sensor. For example, a clonal population of nucleic acids can be attached to a sensor or in the field of a sensor.

In certain configurations, the linker molecule includes at least a first and a second functional group. Generally, the first functional group interacts with a solid support surface (e.g., the attachment moiety on the surface of an electrode, conduction channel or gate of a sensor set forth herein) and the second functional group interacts with the reaction component (e.g. a polymerase, primed template nucleic acid or nucleotide). Exemplary first functional groups include a pyrene, a benzene, a cyclohexane, and 2,3-dichloro-5,6-dicyano-1,4-benzoquinone. An exemplary second functional group is maleimide. In certain embodiments in which the conduction channel is a SWNT, the linker molecule interacts with a sidewall of the SWNT through pi-pi stacking.

A SWNT can be attached to a reaction component (such as polymerases, nucleic acids, nucleotides or the like) using chemistries such as those set forth, for example, in Besterman et al., Nano. Lett. 3, 727 (2003); and Chen et al. Proc. Natl. Acad. Sci. U.S.A. 100, 4984 (2003). A single molecule can be attached to a charge sensor by creating a single covalent defect on the charge sensor. Methods for making a single covalent defect are described, for example, in US Pat. App. Pub. No. 2018/0112265 A1 or 2018/0155773 A1; or Goldsmith et al. Science 315, 77 (2007), each of which is incorporated herein by reference. Using such methods, a SWNT can be produced having a single defect such that a variety of attachment chemistries can be used to link a single reaction component molecule to the reactive defect site selectively, without coating the rest of the SWNT with additional reaction components. SWNTs can also be attached to a single molecule by non-covalent means, for example, using techniques set forth in Chen et al, J. Am. Chem. Soc. 123, 3838 (2001), which is incorporated herein by reference.

A component of a nucleic acid detection reaction can be attached to a sensor such that the reaction component conducts voltage or current between two electrodes. As such, the reaction component can form all or part of a channel that connects a source electrode to a drain electrode. In various configurations of the present disclosure, a sensor includes a polymerase, nucleic acid (e.g. primer or template) or nucleotide connected to both a positive and a negative electrode to complete a circuit. Binding of the attached component to one or more other reaction components can be detected as changes in the current, voltage or other electrical characteristic measured across the circuit. In this configuration the attached reaction component is directly “wired” to both the positive and negative electrodes to complete a circuit. Accordingly, the attached reaction component provides an essential conduction path between electrodes of the sensor. Exemplary methods, reagents and apparatus that can be used to wire electrodes with proteins are set forth in US Pat App. Pub. No. 2018/0305727 A1, which is incorporated herein by reference, and can be modified to wire electrodes with other reaction components set forth herein such as a primer nucleic acid, template nucleic acid or nucleotide.

In various configurations, at least one of voltage or current is initiated in a molecular circuit that includes an immobilized reaction component such as a polymerase, nucleic acid or nucleotide. When another reaction component binds to the wired reaction component, electrical changes in the circuit occur and can be sensed. For example, a polymerase can form a circuit between two electrodes such that association and dissociation of a ternary complex that includes the polymerase, a primed template nucleic acid and next correct nucleotide can be detected. Similarly, a primed template nucleic acid can form a circuit between two electrodes such that association and dissociation of a ternary complex that includes the primed template nucleic acid, a polymerase and next correct nucleotide can be detected. Useful electrical changes, or informative signal states, may include current, voltage, impedance, conductivity, resistance, capacitance, or the like. In some examples, a voltage is initiated in the circuit and then changes in the current through the circuit are measured due reaction components binding to form a complex or due to the complex dissociating. In other examples, a current is initiated in the circuit, and changes to voltage in the circuit are measured due to reaction components being present in as complex or due to dissociation of the complex. In other examples, impedance, conductivity, or resistance is measured. In examples wherein the circuit further includes a gate electrode, at least one of a voltage or current may be applied to the gate electrode, and voltage, current, impedance, conductivity, resistance, or other electrical change in the circuit may be measured to detect association and dissociation of a complex that includes the reaction components.

In particular configurations, at least one linker is attached to a reaction component to provide a physical anchor to a solid support or sensor component such as an electrode, conducting channel or non-conducting sensor surface. Conducting linkers can include any convenient moiety that provides an electrically conductive connection or semi-conducting connection between the reaction component and sensor component (e.g. electrode or channel). Further, linkers may provide extensions, to help span an electrode gap that is wider than the length of the reaction component that is intended to form a circuit between the two electrodes. Such linkers can be selected to provide the advantage of keeping the reaction component away from contacting the electrodes where unfavorable or damaging interactions may occur with the electrodes, such as a denaturing adsorption to the electrode. By way of example, the electrode can include a gold attachment moiety and the linker can include a thiol moiety, such that the thiol moiety of the linker couples to the gold moiety of the electrode via well-known thiol-gold binding. In another embodiment, a linker may present a click-chemistry binding moiety, for coupling to sensor components that are derivatized with the cognate binding partners for the click-chemistry binding group. In various embodiments, a maleimide group on a linker can couple to a surface cysteine on a polymerase or other proteinaceous component of a reaction set forth herein. The cysteine may be native to the polymerase or it may be a point mutation in the primary sequence of the polymerase or it may be in the sequence of a peptide that is fused to the polymerase.

As exemplified above, a linker can provide an uninterrupted chain of covalent bonds between two entities. Alternatively, a linker can include at least one non-covalent bond between the two entities. The presence of non-covalent bond(s) can provide an advantage of reversible formation and dissociation of the linker. Particularly useful non-covalently bonded moieties are the moieties of receptors and ligands that participate in affinity interactions between a receptor and ligand. Exemplary receptor-ligand pairs that can form all or part of a linker include, but are not limited to, streptavidin (or analog thereof) and biotin (or analog thereof), antibody (or functional fragment thereof) and epitope, sense and antisense strands of nucleic acid, sense and antisense strands of protein nucleic acids (PNA), lectin and carbohydrate, aptamers, or peptide binding pairs.

A linker that is intended to create a circuit with components of a sensor can include any of a variety of molecules that provide for conduction of current or voltage. In certain configurations, such linkers can include molecular wires from the many forms known from the art of molecular electronics. For example, linkers can include single stranded DNA, double stranded DNA, peptides, peptide alpha-helices, antibodies or functional fragments thereof, carbon nanotubes, graphene nanoribbons, natural polymers, synthetic polymers, other organic molecules with p-orbitals for electron delocalization, metal or semiconductor nanorods or nanoparticles. Other linkers include, for example, receptor-ligand pairs set forth herein, a peptide nucleic acid (PNA) duplex, a PNA-DNA hybrid duplex, a protein alpha-helix, a graphene-like nanoribbon, a natural polymer, a synthetic organic molecule (e.g. a synthetic polymer), and an antibody Fab domain.

Linkers can be chosen for any of a variety of characteristics. For example, the length and flexibility of a linker can be selected to accommodate a desired level of local mobility for a reaction component that is linked to a sensor or other solid support surface. A linker that is relatively short and/or rigid can be beneficial for maintaining a linked reaction component in a relatively small volume of space, for example, to maintain the reaction component in the field of a FET sensor or otherwise near a sensor. A linker can have a length of at least 0.1 nm, 1 nm, 5 nm, 10 nm, 50 nm, 100 nm, 500 nm, 1 μm, 2 μm or more. Longer linkers can provide for an increased range of motion and larger volume of space for reaction to occur. Accordingly, a linker can have a length of at most 2 μm, 1 μm, 500 nm, 100 nm, 50 nm, 10 nm, 5 nm, 1 nm, 0.1 nm or less. A linker can have a length that is between any of these exemplary upper limit values and lower limit values.

In particular configurations, a reaction component can be directly attached to a sensor or to a solid support surface that is in proximity to a sensor. The attachment can be covalent or non-covalent. A linker can be as short as a single covalent bond between the native structure of the reaction component and the attachment site on the sensor or other solid support surface. An advantage of short linkers includes minimizing the length of the conduction path, since the parts of the conduction path outside of the reaction component can be sources of unwanted noise, resistance or capacitance. Of course, multiple linkages, each linkage having one or more bonds, can attach a reaction component to a sensor or other solid support surface.

In particular embodiments, a sensor is configured for detection at a single molecule level. For example, a sensor component can be configured to be attached to no more than a single molecule of a particular type. More specifically, a sensor component can be attached to one and only one polymerase molecule, a sensor can be attached to one and only one primed template nucleic acid molecule, a sensor can be attached to one and only one primer nucleic acid molecule, a sensor can be attached to one and only one template nucleic acid molecule, or a sensor can be attached to one and only one nucleotide molecule. It will be understood that a sensor or sensor component can be attached to several different types of molecules, albeit that only one molecule of each type is attached.

Alternatively, a sensor can be configured to detect at an ensemble level. Accordingly, an ensemble of molecules of the same type can be immobilized at or near a sensor and reactions that involve two or more members of the ensemble can be detected. For example, a sensor component can be attached to an ensemble of polymerase molecules, a sensor can be attached to an ensemble of primed template nucleic acid molecules, a sensor can be attached to an ensemble of primer nucleic acid molecules, a sensor can be attached to an ensemble of template nucleic acid molecules, or a sensor can be attached to an ensemble of nucleotide molecules.

The mean spacing between electrodes can be selected to suit a particular use of a sensor set forth herein. For example, this spacing can be at least 1 nm, 5 nm, 10 nm, 50 nm, 100 nm, 500 nm, 1 μm, 10 μm or more. Alternatively or additionally, the spacing can be at most 10 μtm, 1 μm, 500 nm, 100 nm, 50 nm, 10 nm, 5 nm, 1 nm or less.

In particular configurations, multiple sensors are fabricated and/or used in parallel. The sensors can be attached to the same type of reaction component (e.g. each sensor being attached to a polymerase of the same type or to a primer nucleic acid having a common sequence) or each sensor can be attached to a different type of reaction component (e.g. each sensor being attached to a template nucleic acid having a different sequence). An apparatus of the present disclosure can include an array of sensors that include at least 1, 10, 100, 1×10³, 1×10⁴, 1×10⁵, 1×10⁶ or more individual sensors. Alternatively or additionally, an apparatus of the present disclosure can include an array of sensors that include at most 1×10⁶, 1×10⁵, 1×10⁴, 1×10³, 100, 1 or fewer individual sensors.

The present disclosure provides attachment modalities that facilitate formation of a ternary complex at a position that is proximal to a sensor. This can be achieved by attaching the polymerase component of the complex in proximity to the sensor or by attaching the nucleotide component of the complex in proximity to the sensor. In these two configurations, the 3′ end of the primer component of the primed template nucleic acid will localize near the sensor due to its affinity for the component of the complex that is immobilized. In configurations where, the polymerase or nucleotide is not immobilized, for example in configurations where the primed template nucleic acid is immobilized, it will be beneficial to localize the 3′ end of the primer at a location where formation of a ternary complex will be accurately detected. For example, a nucleotide that is incorporated at the 3′ end can include an affinity moiety that is attracted to the sensor. Useful affinity moieties include, but are not limited to receptors, ligands, charged moieties, magnetic moieties or the like.

In particular configurations, it can also be beneficial to prevent long strands of nucleic acid from directly contacting certain components of a sensor. This can minimize risk of short circuiting the sensor or from experiencing damage when contacting the sensor. An exemplary technique for minimizing this risk is to degrade double stranded DNA that is produced during a sequencing reaction while maintaining a relatively short primer region and while maintaining the single stranded template region that is to be sequenced. For example, a primed template nucleic acid that is being sequenced can be treated with a double stranded specific exonuclease in the presence of polymerase. This will result in degradation of double stranded portions of the nucleic acid that have already been sequenced or that are not to be sequenced. However, the single stranded region will not be degraded due to specificity of the double stranded exonuclease and the double stranded primer region will be protected from degradation by the bound polymerase. In another configuration, an RNA primer can be extended along a DNA template by reverse transcriptase. The RNA strand that extends during a sequencing reaction can be degraded using an RNA endonuclease. For example, HIV reverse transcriptase can degrade the RNA strand.

### Polymerases

Any of a variety of polymerases can be used in a method or apparatus set forth herein, for example, to form a stabilized ternary complex or to carry out primer extension. Polymerases that may be used include naturally occurring polymerases and modified variations thereof, including, but not limited to, mutants, recombinants, fusions, genetic modifications, chemical modifications, synthetics, and analogs. Naturally occurring polymerases and modified variations thereof are not limited to polymerases that have the ability to catalyze a polymerization reaction. Optionally, the naturally occurring and/or modified variations thereof have the ability to catalyze a polymerization reaction in at least one condition that is not used during formation or examination of a stabilized ternary complex. Optionally, the naturally-occurring and/or modified variations that participate in stabilized ternary complexes have modified properties, for example, enhanced binding affinity to nucleic acids, reduced binding affinity to nucleic acids, enhanced binding affinity to nucleotides, reduced binding affinity to nucleotides, enhanced specificity for next correct nucleotides, reduced specificity for next correct nucleotides, reduced catalysis rates, catalytic inactivity etc. Mutant polymerases include, for example, polymerases wherein one or more amino acids are replaced with other amino acids, or insertions or deletions of one or more amino acids. Exemplary polymerase mutants that can be used to form a stabilized ternary complex include, for example, those set forth in US Pat. App. Pub. Nos. 2017/0314072 A1 or US 2018/0155698, each of which is incorporated herein by reference.

Modified polymerases include polymerases that contain an exogenous label moiety (e.g., an exogenous charge label), which can be used to detect the polymerase. Optionally, the label moiety can be attached after the polymerase has been at least partially purified using protein isolation techniques. For example, the exogenous label moiety can be covalently linked to the polymerase using a free sulfhydryl or a free amine moiety of the polymerase. This can involve covalent linkage to the polymerase through the side chain of a cysteine residue, or through the free amino group of the N-terminus. An exogenous label moiety can also be attached to a polymerase via protein fusion. Exemplary label moieties that can be attached via protein fusion include, for example, affinity peptides, receptor proteins or peptide ligands.

Alternatively, a polymerase that participates in a stabilized ternary complex, or that is used to extend a primer need not be attached to an exogenous label. For example, the polymerase need not be covalently attached to an exogenous label. Instead, the polymerase can lack any exogenous label until it associates with a labeled nucleotide and/or labeled nucleic acid (e.g. labeled primer and/or labeled template).

Different activities of polymerases can be exploited in a method set forth herein. A polymerase can be useful, for example, in a primer extension step, examination step or both steps. The different activities can follow from differences in the structure (e.g. via natural activities, mutations or chemical modifications). Nevertheless, polymerase can be obtained from a variety of known sources and applied in accordance with the teachings set forth herein and recognized activities of polymerases. Useful DNA polymerases include, but are not limited to, bacterial DNA polymerases, eukaryotic DNA polymerases, archaeal DNA polymerases, viral DNA polymerases and phage DNA polymerases. Bacterial DNA polymerases include E. coli DNA polymerases I, II and III, IV and V, the Klenow fragment of E. coli DNA polymerase, Clostridium stercorarium (Cst) DNA polymerase, Clostridium thermocellum (Cth) DNA polymerase and Sulfolobus solfataricus (Sso) DNA polymerase. Eukaryotic DNA polymerases include DNA polymerases α, β, γ, δ, €, η, μ, σ, μ, and k, as well as the Revl polymerase (terminal deoxycytidyl transferase) and terminal deoxynucleotidyl transferase (TdT). Viral DNA polymerases include T4 DNA polymerase, phi-29 DNA polymerase, GA-1, phi-29-like DNA polymerases, PZA DNA polymerase, phi-15 DNA polymerase, Cpl DNA polymerase, Cpl DNA polymerase, T7 DNA polymerase, and T4 polymerase. Other useful DNA polymerases include thermostable and/or thermophilic DNA polymerases such as Thermus aquaticus (Taq) DNA polymerase, Thermus filiformis (Tfi) DNA polymerase, Thermococcus zilligi (Tzi) DNA polymerase, Thermus thermophilus (Tth) DNA polymerase, Thermus flavusu (Tfl) DNA polymerase, Pyrococcus woesei (Pwo) DNA polymerase, Pyrococcus furiosus (Pfu) DNA polymerase and Turbo Pfu DNA polymerase, Thermococcus litoralis (Tli) DNA polymerase, Pyrococcus sp. GB-D polymerase, Thermotoga maritima (Tma) DNA polymerase, Bacillus stearothermophilus (Bst) DNA polymerase, Pyrococcus Kodakaraensis (KOD) DNA polymerase, Pfx DNA polymerase, Thermococcus sp. JDF-3 (JDF-3) DNA polymerase, Thermococcus gorgonarius (Tgo) DNA polymerase, Thermococcus acidophilium DNA polymerase; Sulfolobus acidocaldarius DNA polymerase; Thermococcus sp. go N-7 DNA polymerase; Pyrodictium occultum DNA polymerase; Methanococcus voltae DNA polymerase; Methanococcus thermoautotrophicum DNA polymerase; Methanococcus jannaschii DNA polymerase; Desulfurococcus strain TOK DNA polymerase (D. Tok Pol); Pyrococcus abyssi DNA polymerase; Pyrococcus horikoshii DNA polymerase; Pyrococcus islandicum DNA polymerase; Thermococcus fumicolans DNA polymerase; Aeropyrum pernix DNA polymerase; and the heterodimeric DNA polymerase DP1/DP2. Engineered and modified polymerases also are useful in connection with the disclosed techniques. For example, modified versions of the extremely thermophilic marine archaea Thermococcus species 9° N (e.g., Therminator DNA polymerase from New England BioLabs Inc.; Ipswich, MA) can be used. Still other useful DNA polymerases, including the 3PDX polymerase are disclosed in U.S. Pat. No. 8,703,461, the disclosure of which is incorporated herein by reference.

Useful RNA polymerases include, but are not limited to, viral RNA polymerases such as T7 RNA polymerase, T3 polymerase, SP6 polymerase, and Kll polymerase; Eukaryotic RNA polymerases such as RNA polymerase I, RNA polymerase II, RNA polymerase III, RNA polymerase IV, and RNA polymerase V; and Archaea RNA polymerase.

Another useful type of RNA polymerase is a primase. Primase catalyzes the synthesis of a short RNA or DNA primer that is complementary to a single-stranded DNA template. A primase can be used to synthesize a primer to be used in a nucleic acid analysis method set forth herein. One or more nucleotides that are incorporated into a primer by a primase (or other polymerase) can include an attachment moiety that can be reacted with solid support, sensor or other entity to attach the primer. Exemplary primases include, for example, primases that belong to the archaea-eukaryotic primase (AEP) superfamily such as Human PrimPol or archaeal PriS, or DnaG-type primases such as LigD.

Reverse transcriptase is another useful type of polymerase Exemplary reverse transcriptases include, but are not limited to, HIV-1 reverse transcriptase from human immunodeficiency virus type 1 (PDB 1HMV), HIV-2 reverse transcriptase from human immunodeficiency virus type 2, M-MLV reverse transcriptase from the Moloney murine leukemia virus, AMV reverse transcriptase from the avian myeloblastosis virus, and Telomerase reverse transcriptase that maintains the telomeres of eukaryotic chromosomes.

A polymerase having an intrinsic 3′-5′ proofreading exonuclease activity can be useful for some embodiments. Polymerases that substantially lack 3′-5′ proofreading exonuclease activity are also useful in some embodiments, for example, in most genotyping and sequencing embodiments. Absence of exonuclease activity can be a wild type characteristic or a characteristic imparted by a variant or engineered polymerase structure. For example, exo minus Klenow fragment is a mutated version of Klenow fragment that lacks 3′-5′ proofreading exonuclease activity. Klenow fragment and its exo minus variant can be useful in a method or composition set forth herein.

A polymerase can associate (covalently or non-covalently) with a DNA clamp protein. A DNA clamp, also known as a sliding clamp, is a protein fold that serves as a processivity-promoting factor in DNA replication. A clamp protein can bind to polymerase, thereby preventing the polymerase from dissociating from the template DNA strand. A DNA clamp can be attached at or near a sensor to provide a moiety for localizing a polymerase at or near the sensor for accurate signal detection. A DNA clamp can be used in addition to, or as an alternative to, linkers set forth herein as useful for immobilizing DNA polymerase. For example, A DNA clamp can provide a useful modification to the configuration shown in FIG. 2, 5 or 7. Exemplary DNA clamps, methods for obtaining the claims and methods for their use that can be modified in accordance with the disclosure herein are set forth in Bruck et al. Genome Biol. 2 (1): reviews 3001.1-3001.3 (2001) or Matsumiya et al. Protein Sci. 10 (1): 17-23 (2001), each of which is incorporated herein by reference.

### Nucleic Acids

Nucleic acid templates that are used in a method or composition herein can be DNA such as genomic DNA, synthetic DNA, amplified DNA, complementary DNA (cDNA) or the like. RNA can also be used such as mRNA, ribosomal RNA, tRNA or the like. Nucleic acid analogs can also be used as templates herein. Thus, a mixture of nucleic acids used herein can be derived from a biological source, synthetic source or amplification product. Primers used herein can be DNA, RNA or analogs thereof.

Exemplary organisms from which nucleic acids can be derived include, for example, those from a mammal such as a rodent, mouse, rat, rabbit, guinea pig, ungulate, horse, sheep, pig, goat, cow, cat, dog, primate, human or non-human primate; a plant such as Arabidopsis thaliana, corn, sorghum, oat, wheat, rice, canola, or soybean; an algae such as Chlamydomonas reinhardtii; a nematode such as Caenorhabditis elegans; an insect such as Drosophila melanogaster, mosquito, fruit fly, honey bee or spider; a fish such as zebrafish; a reptile; an amphibian such as a frog or Xenopus laevis; a dictyostelium discoideum; a fungi such as Pneumocystis carinii, Takifugu rubripes, yeast, Saccharamoyces cerevisiae or Schizosaccharomyces pombe; or a Plasmodium falciparum. Nucleic acids can also be derived from a prokaryote such as a bacterium, Escherichia coli, staphylococci or Mycoplasma pneumoniae; an archae; a virus such as Hepatitis C virus or human immunodeficiency virus; or a viroid. Nucleic acids can be derived from a homogeneous culture or population of the above organisms or alternatively from a collection of several different organisms, for example, in a community or ecosystem. Nucleic acids can be isolated using methods known in the art including, for example, those described in Sambrook et al., Molecular Cloning: A Laboratory Manual, 3rd edition, Cold Spring Harbor Laboratory, New York (2001) or in Ausubel et al., Current Protocols in Molecular Biology, John Wiley and Sons, Baltimore, Md. (1998), each of which is incorporated herein by reference.

A template nucleic acid can be obtained from a preparative method such as genome isolation, genome fragmentation, gene cloning and/or amplification. The template can be obtained from an amplification technique such as polymerase chain reaction (PCR), rolling circle amplification (RCA), multiple displacement amplification (MDA) or the like. Exemplary methods for isolating, amplifying and fragmenting nucleic acids to produce templates for analysis on an array are set forth in U.S. Pat. Nos. 6,355,431 or 9,045,796, each of which is incorporated herein by reference. Amplification can also be carried out using a method set forth in Sambrook et al., Molecular Cloning: A Laboratory Manual, 3rd edition, Cold Spring Harbor Laboratory, New York (2001) or in Ausubel et al., Current Protocols in Molecular Biology, John Wiley and Sons, Baltimore, Md. (1998), each of which is incorporated herein by reference.

A template nucleic acid can be obtained from a preparative method such as genome isolation, genome fragmentation, gene cloning and/or amplification. The template can be obtained from an amplification technique such as polymerase chain reaction (PCR), rolling circle amplification (RCA), multiple displacement amplification (MDA) or the like. Exemplary methods for isolating, amplifying and fragmenting nucleic acids to produce templates for analysis on an array are set forth in U.S. Pat. Nos. 6,355,431 or 9,045,796, each of which is incorporated herein by reference. Amplification can also be carried out using a method set forth in Sambrook et al., Molecular Cloning: A Laboratory Manual, 3rd edition, Cold Spring Harbor Laboratory, New York (2001) or in Ausubel et al., Current Protocols in Molecular Biology, John Wiley and Sons, Baltimore, Md. (1998), each of which is incorporated herein by reference.

### Nucleotides

A stabilized ternary complex can include a native nucleotide, nucleotide analog or modified nucleotide as desired to suit a particular application or configuration of the methods set forth herein. Optionally, a nucleotide analog has a nitrogenous base, five-carbon sugar, and phosphate group, wherein any moiety of the nucleotide may be modified, removed and/or replaced as compared to a native nucleotide. Nucleotide analogs may be non-incorporable nucleotides (i.e. nucleotides that are incapable of reacting with the 3′ oxygen of a primer to form a covalent linkage). Such nucleotides that are incapable of incorporation include, for example, monophosphate and diphosphate nucleotides. In another example, the nucleotide may contain modification(s) to the triphosphate group that render the nucleotide non-incorporable. Examples of non-incorporable nucleotides may be found in U.S. Pat. No. 7,482,120, which is incorporated by reference herein. In some embodiments, non-incorporable nucleotides may be subsequently modified to become incorporable. Non-incorporable nucleotide analogs include, but are not limited to, alpha-phosphate modified nucleotides, alpha-beta nucleotide analogs, beta-phosphate modified nucleotides, beta-gamma nucleotide analogs, gamma-phosphate modified nucleotides, or caged nucleotides. Further examples of nucleotide analogs are described in U.S. Pat. No. 8,071,755, which is incorporated by reference herein. Moieties that are attached to the moiety of a nucleotide can provide a dual function of inhibiting incorporation and of providing an electrically detectable label. Examples of nucleotides having electrically detectable labels at the 5′ position are set forth in WO 2018/026855 A1, which is incorporated herein by reference.

Nucleotide analogs that are used herein, for example, to participate in stabilized ternary complexes can include terminators that reversibly prevent subsequent nucleotide incorporation at the 3′-end of the primer after the analog has been incorporated into the primer. For example, U.S. Pat. Nos. 7,544,794 and 8,034,923 (the disclosures of these patents are incorporated herein by reference) describe reversible terminators in which the 3′-OH group is replaced by a 3′-ONH₂ moiety. Another type of reversible terminator is linked to the nitrogenous base of a nucleotide as set forth, for example, in U.S. Pat. No. 8,808,989 (the disclosure of which is incorporated herein by reference). Other reversible terminators that similarly can be used in connection with the methods described herein include those described in references cited elsewhere herein or in U.S. Pat. Nos. 7,956,171, 8,071,755, and 9,399,798 (the disclosures of these U.S. patents are incorporated herein by reference). In certain embodiments, a reversible terminator moiety can be removed from a primer, in a process known as “deblocking,” allowing for subsequent nucleotide incorporation. Compositions and methods for deblocking are set forth in references cited herein in the context of reversible terminators.

Alternatively, nucleotide analogs irreversibly prevent nucleotide incorporation at the 3′-end of the primer to which they have been incorporated. Irreversible nucleotide analogs include 2′, 3′-dideoxynucleotides (ddNTPs such as ddGTP, ddATP, ddTTP, ddCTP). Dideoxynucleotides lack the 3′-OH group of dNTPs that would otherwise participate in polymerase-mediated primer extension. Thus, the 3′ position has a hydrogen moiety instead of the native hydroxyl moiety. Irreversibly terminated nucleotides can be particularly useful for genotyping applications or other applications where primer extension or sequential detection along a template nucleic acid is not desired.

In particular embodiments, nucleotide analogs that are used herein, for example, to participate in stabilized ternary complexes do not include blocking groups (e.g. reversible terminators) that prevent subsequent nucleotide incorporation at the 3′-end of the primer after the analog has been incorporated into the primer. This can be the case whether or not an extension step is carried out using nucleotide(s) having a blocking group (e.g. reversible terminator).

In some embodiments, a nucleotide that is used herein, for example, to participate in forming a stabilized ternary complex, can include an exogenous label. For example, an exogenously labeled nucleotide can include a reversible or irreversible terminator moiety, an exogenously labeled nucleotide can be non-incorporable, an exogenously labeled nucleotide can lack terminator moieties, an exogenously labeled nucleotide can be incorporable or an exogenously labeled nucleotide can be both incorporable and non-terminated. Exogenously labeled nucleotides can be particularly useful when used to form a stabilized ternary complex with a non-labeled polymerase.

Alternatively, a nucleotide that is used herein, for example, to participate in forming a ternary complex can lack exogenous labels (i.e. the nucleotide can be “non-labeled”). For example, a non-labeled nucleotide can include a reversible or irreversible terminator moiety, a non-labeled nucleotide can be non-incorporable, a non-labeled nucleotide can lack terminator moieties, a non-labeled nucleotide can be incorporable, or a non-labeled nucleotide can be both incorporable and non-terminated. Non-labeled nucleotides can be useful when a label on a polymerase is used to detect a stabilized ternary complex or when label-free detection is used. Non-labeled nucleotides can also be useful in an extension step of a method set forth herein. It will be understood that absence of a moiety or function for a nucleotide refers to the nucleotide having no such function or moiety. However, it will also be understood that one or more of the functions or moieties set forth herein for a nucleotide, or analog thereof, or otherwise known in the art for a nucleotide, or analog thereof, can be specifically omitted in a method or composition set forth herein.

Optionally, a nucleotide (e.g. a native nucleotide or nucleotide analog) is present in a mixture during or after formation of a stabilized ternary complex. For example, at least 1, 2, 3, 4 or more nucleotide types can be present. Alternatively or additionally, at most 4, 3, 2, or 1 nucleotide types can be present. Similarly, one or more nucleotide types that are present can be complementary to at least 1, 2, 3 or 4 base types in a template nucleic acid. Alternatively or additionally, one or more nucleotide types that are present can be complementary to at most 4, 3, 2, or 1 base types in a template nucleic acid. Different base types can be identifiable by the presence of different exogenous labels on the different nucleotides. Alternatively, two or more nucleotide types can have exogenous labels that are not distinguishable. In the latter format the different nucleotides can nevertheless be distinguished due to being separately delivered to a sensor or due to an encoding and decoding scheme as set forth, for example, in US Pat. App. Pub. No. 2018/0305749 A1 or U.S. Pat. No. 9,951,385, each of which is incorporated herein by reference.

In some configurations, a nucleotide can include a moiety that has affinity for a component of a charge sensor. For example, the affinity moiety can include a receptor having affinity for a ligand that is attached to the charge sensor. Alternatively, the affinity moiety can include a ligand having affinity for a ligand that is attached to the charge sensor. Nucleotides having an affinity moiety can be incorporated into a primer, for example, during the extension step of a sequencing reaction. The presence of the affinity moiety can be particularly useful for maintaining localization of the primer 3′ end in proximity to the sensor even as the primer is extended. This localization mechanism in turn maintains the site of ternary complex formation in proximity to the sensor, thereby favoring accuracy of detection. This localization mechanism can be particularly useful for sensor configurations where polymerase and nucleotide are not immobilized such as the configuration exemplified in FIG. 1.

### Labels

A reaction component that is to be detected using an electronic sensor can include a label moiety. In cases where the reaction is formation of a stabilized ternary complex, one or more components that are capable of participating in the stabilized ternary complex can be labeled. A label can be attached to a nucleotide, polymerase or other molecule via a linker. A linker that is present in a nucleotide or polymerase can be, but need not be, cleavable. For example, the linker can be stable to conditions used in methods set forth herein such that the covalent structure of the linker is not changed during any particular step, or throughout all steps, of a method set forth herein. A linker that is present in a nucleotide analog can be at least as chemically stable as one or more other moieties in the analog under the conditions that the nucleotide is used. For example, the linker can be as chemically stable as the nitrogenous base, sugar and/or phosphate moiety during any particular step, or throughout all steps, of a method set forth herein.

A label can be attached to a polymerase, for example, via linkage through a reactive amino acid side chain of the polymerase. Alternatively or additionally, a label can be attached to a primed template nucleic acid via linkage to the primer or the template. For example, the label can be attached to the base moiety, sugar moiety or phosphate moiety of a nucleic acid. Moreover, a label can be attached to a nucleotide, for example, via linkage to the base moiety, sugar moiety or phosphate moiety. In some configurations, a nucleotide that is used herein does not include a label moiety at the 5′ position nor on the phosphate moiety.

A reaction that is monitored or detected in a method set forth herein need not rely on removal of a label. In this regard, a label need not be present and if a label is present it need not be detected nor does removal of the label need to be detected.

A label can be attached to a reaction component via a linker that is not cleaved or removed during the course of one or more steps of a method set forth herein. Alternatively, a label can be attached to a reaction component via cleavable linker. For example, a label can be attached to a nucleotide via the 5′ phosphate moiety such that the label can be removed when the nucleotide is incorporated into a primer. Alternatively, a label is attached to a nucleotide at a location other than the 5′ position of the sugar. For example, a label can be attached to the base position, 3′ position of the sugar or 2′ position of the sugar. Attachment of a label to the 2′ or 3′ position of the nucleotide sugar at the 3′ end of a primer can provide a means to block the primer from being extended by addition of a subsequent nucleotide. Again, a label that is attached at the 2′ position, 3′ position or other position of a nucleotide can be attached via a cleavable linker or via a non-cleavable linker. Use of a cleavable linker can provide for cyclic reversible extension in a sequencing embodiment as set forth in further detail below.

A particularly useful label will have a charge state (e.g., +1, +2, or −1, −2, etc.) that produces a detectable signal state from a sensor herein. For example, a label moiety can include a sulfonate group (—S0₃-, with −1 charge), or a quaternary ammonium substituent (—R₃N⁺ with a +1 charge). In certain configurations, a linker provides a range of motion that allows a label moiety to come into close proximity to a conducting channel or other component of a sensor, for example, when interacting with other reaction components that form a stabilized ternary complex. In the case of nucleotides, useful labels include, but are not limited to, a chlorine substitution at the 6-position of purine bases, a sulfoxide present at the 5′ position of the sugar, a sulfoxide derivatization of the 2-position of a pyrimidine base or a bromine substitution at the 5-position of a pyrimidine base.

Other useful label moieties include, but are not limited to, chemical moieties that provide a polarity (e.g., —C═O, —OH functionality), non-polar character (e.g., hydrophobicity through use of non-polar functionality like —CHₙ), or steric effects (e.g., large fused ring systems). Particularly useful labels are capable of providing enhanced signaling according to any of a variety of mechanisms. For example, enhanced signaling can arise through direct interaction of negative charges or positive charges on a label with the conducting portion of an electronic sensor; through direct n-n and hydrophobic interaction of aromatic rings, provided by a label, with the conducting portion of an electronic sensor; through electrochemical reduction and oxidation of the label (e.g., for reversibly oxidizable groups such as ferrocene or hydroquinone; through creating and breaking a conductive link between two conductors within a label or between labels; or through steric interaction of the label that alters the interaction of a stabilized ternary complex with a sensor. Other labels that can be used are set forth in US Pat App. Pub. No. 2018/0112265 A1 or PCT Pub. No. WO 2018/026855 A1, each of which is incorporated herein by reference.

A secondary label can be used in a method of the present disclosure. A secondary label is a binding moiety that can bind specifically to a partner moiety. For example, a ligand moiety can be attached to a polymerase, nucleic acid or nucleotide to allow detection via specific affinity for a receptor. The receptor can optionally be labeled. Exemplary pairs of binding moieties that can be used include, without limitation, antigen and immunoglobulin or active fragments thereof, such as FAbs;

immunoglobulin and immunoglobulin (or active fragments, respectively); avidin and biotin, or analogs thereof having specificity for avidin; streptavidin and biotin, or analogs thereof having specificity for streptavidin; or carbohydrates and lectins.

In some embodiments, the secondary label can be a chemically modifiable moiety. In this embodiment, labels having reactive functional groups can be incorporated into a stabilized ternary complex. Subsequently, the functional group can be covalently reacted with a primary label moiety. Suitable functional groups include, but are not limited to, amino groups, carboxy groups, maleimide groups, oxo groups, groups that participate in click reactions and thiol groups.

In particular configurations a label, or linker that attaches a label to a reaction component, can include a receptor, ligand or other affinity group. An affinity group can promote the positioning and residence of a label on a reaction component to have larger impact on a sensor than may occur when the label is free to move away from the sensor. Exemplary affinity groups and techniques that can be used to position a label in proximity to a sensor using affinity groups include those set forth above in the context of secondary labels or in US Pat App. Pub. No. 2018/0112265 A1 or PCT Pub. No. WO 2018/026855 A1, each of which is incorporated herein by reference.

An affinity group can be highly specific, such as a single stranded DNA 5-mer that would have affinity to its complementary portion of a DNA oligo present in or on a conducting channel of a sensor, or it can just represent a charge affinity, such as a negative charge on the nucleotide being attracted to a positive charge on the conducting channel. Complementary oligos including DNA analogs may also be beneficial as affinity groups. For example, such oligos can include RNA, PNA (peptide nucleic acid) or LNA (locked nucleic acid) in place of native DNA. In the example of a graphene conducting channel or linker, an affinity moiety can include a pyrene which has affinity for the channel via pi-pi stacking interaction of pyrene and graphene. Other affinity groups can include binding peptides, aptamers, interacting proteins such as two components of a protein complex, a small molecule or peptide antigen and a cognate antibody, or other receptor ligand pairs set forth herein or otherwise known in the art. In certain examples, different affinity groups can be present on nucleotides having different bases. As such, different positioning afforded by the affinity group of each nucleotide with respect to a charge sensor can provide distinguishable detection of the different nucleotide types.

### Detection Methods

Provided here is a method of detecting formation of a ternary complex between a polymerase, primer template nucleic acid and next correct nucleotide for the template. The method includes providing a sensor that is configured to include at least one of the reaction components that is immobilized at or near the sensor. The immobilized reaction component can be contacted with the other two components to form a ternary complex. The ternary complex can be detected by measuring a signal state of the sensor that is indicative of formation or presence of the ternary complex.

In particular configurations, current fluctuations result from formation of a ternary complex. The current fluctuations can consist of simple increases and decreases in a square-edged pattern. Alternately, the fluctuations can include any wavelet including shapes that are triangular, sinusoidal, or having any number of Fourier components. The amplitudes, durations, and shapes of these wavelets all encode the activity of the reaction being monitored and therefore can be analyzed using a computer to uncover the presence or absence of ternary complex or to uncover other characteristics such as kinetics of ternary complex association and dissociation.

The present disclosure provides a method for identifying the next correct nucleotide for a primed template nucleic acid. The method can include steps of (a) providing a sensor that includes a first electrode, a second electrode, a conduction channel operably connecting the first electrode to the second electrode and a primed template nucleic acid that is immobilized at the sensor; (b) contacting the primed template nucleic acid with a polymerase and the next correct nucleotide, thereby forming a stabilized ternary complex that includes the primed template nucleic acid, the polymerase and the next correct nucleotide, wherein the stabilized ternary complex is prevented from covalently incorporating the next correct nucleotide into the primed template nucleic acid; and (c) monitoring the sensor to detect a signal produced by the stabilized ternary complex, whereby the next correct nucleotide is identified from the signal.

In an alternative configuration, a method for identifying the next correct nucleotide for a primed template nucleic acid can include steps of (a) providing a sensor that includes a first electrode, a second electrode, a conduction channel operably connecting the first electrode to the second electrode and a polymerase that is immobilized at the sensor; (b) contacting the polymerase with a primed template nucleic acid and the next correct nucleotide, thereby forming a stabilized ternary complex that includes the primed template nucleic acid, the polymerase and the next correct nucleotide, wherein the stabilized ternary complex is prevented from covalently incorporating the next correct nucleotide into the primed template nucleic acid; and (c) monitoring the sensor to detect a signal produced by the stabilized ternary complex, whereby the next correct nucleotide is identified from the signal.

A method for identifying the next correct nucleotide for a primed template nucleic acid can also include steps of (a) providing a sensor having a first electrode that is attached to a polymerase via a first conductive linker and a second electrode that is attached to a primed template nucleic acid via a second conductive linker; (b) contacting the polymerase and the primed template nucleic acid with the next correct nucleotide, thereby forming a stabilized ternary complex including the primed template nucleic acid, the polymerase and the next correct nucleotide, wherein the stabilized ternary complex is prevented from covalently incorporating the next correct nucleotide into the primed template nucleic acid; and (c) monitoring the sensor to detect a circuit between the first and second electrode produced by the stabilized ternary complex, whereby the next correct nucleotide is identified from the signal.

In other embodiments, a method for identifying the next correct nucleotide for a primed template nucleic acid can include steps of (a) providing a sensor having a first electrode that is attached to a polymerase via a first conductive linker and a second electrode that is attached to a nucleotide via a second conductive linker; (b) contacting the polymerase and the nucleotide with a primed template nucleic acid, thereby forming a stabilized ternary complex including the primed template nucleic acid, the polymerase and the nucleotide, wherein the stabilized ternary complex is prevented from covalently incorporating the nucleotide into the primed template nucleic acid; and (c) monitoring the sensor to detect a circuit between the first and second electrode produced by the stabilized ternary complex, whereby the nucleotide is identified as the next correct nucleotide for the primed template nucleic acid.

Alternatively, a method for identifying the next correct nucleotide for a primed template nucleic acid can include steps of (a) providing a sensor having a first electrode that is attached to a primed template nucleic acid via a first conductive linker and a second electrode that is attached to a nucleotide via a second conductive linker; (b) contacting the primed template nucleic acid and the nucleotide with a polymerase, thereby forming a stabilized ternary complex including the primed template nucleic acid, the polymerase and the nucleotide, wherein the stabilized ternary complex is prevented from covalently incorporating the nucleotide into the primed template nucleic acid; and (c) monitoring the sensor to detect a circuit between the first and second electrode produced by the stabilized ternary complex, whereby the nucleotide is identified as the next correct nucleotide for the primed template nucleic acid.

As exemplified by the methods set forth above and elsewhere herein, a method of detecting or characterizing a nucleic acid can include a step of contacting a polymerase, primed template nucleic acid and nucleotide to form a stabilized ternary complex. At least one of the components that is capable of participating in a ternary complex will generally be immobilized at or near a sensor. The other components that are capable of participating in the ternary complex can be provided to the immobilized component under conditions for forming the ternary complex. In exemplary configurations, a first component can be immobilized at or near the sensor and one or both of the second and third components can be delivered to the immobilized component in fluid phase and under conditions for forming the stabilized ternary complex. By way of more specific example, a polymerase can be immobilized at or near a sensor while a primed template nucleic acid or nucleotide, or both are delivered to the polymerase in fluid phase. Similarly, a primed template nucleic acid can be immobilized at or near a sensor while a polymerase or nucleotide, or both are delivered to the primed template nucleic acid in fluid phase. Alternatively, a nucleotide can be immobilized at or near a sensor while a primed template nucleic acid or polymerase, or both are delivered to the nucleotide in fluid phase.

In some configurations, a first component that is capable of participating in a ternary complex is immobilized at or near a sensor in a way that presence or absence of the ternary complex can be distinguished by the sensor. In exemplary configurations, a first component can be immobilized at or near the sensor and one or both of the second and third components can also be immobilized at or near the sensor. The immobilized components can be allowed to interact under conditions for forming a stabilized ternary complex. For example, a non-immobilized component that is capable of combining with two immobilized components to form a ternary complex can be delivered in fluid phase to the immobilized components. By way of more specific example, a polymerase can be immobilized at or near a sensor while a primed template nucleic acid or nucleotide, or both are immobilized in sufficient proximity to the polymerase in order to form a stabilized ternary complex the polymerase in fluid phase. Similarly, a primed template nucleic acid can be immobilized at or near a sensor while a polymerase or nucleotide, or both are immobilized in sufficient proximity to the primed template nucleic acid in order to form a stabilized ternary complex the polymerase in fluid phase. Alternatively, a nucleotide can be immobilized at or near a sensor while a primed template nucleic acid or polymerase, or both are immobilized in sufficient proximity to the nucleotide in order to form a stabilized ternary complex the polymerase in fluid phase.

In the above exemplary configurations, a first component that is capable of participating in a stabilized ternary complex can be immobilized at or near a sensor as set forth in the description of various sensor apparatus herein. For example, the first component can be attached to a sensor via a conducting linker or non-conducting linker to suit a particular sensing technique. One or more other components that are capable of forming a ternary complex with the first component can be attached to the sensor via a conducing linker or via a non-conducting linker. Indeed, the one or more other components can be attached to a solid support surface that is proximal to the sensor but that does not allow conduction of current or voltage from the sensor to the one or more other component.

Particular configurations of the methods can be used to perform cyclical reactions such as sequencing of nucleic acids. Each cycle can include delivering reagents for a sequencing reaction to a flow cell or other vessel where, optionally, the reaction, or products of the reaction, will be observed using a sensor set forth herein. A particularly useful sequencing reaction is a Sequencing By Binding™ (SBB™) reaction such as those described in commonly owned US Pat. App. Pub. Nos. 2017/0022553 A1, 2018/0044727 A1, 2018/0187245 A1 or 2018/0208983 A1, each of which is incorporated herein by reference. Generally, methods for determining the sequence of a template nucleic acid molecule can be based on formation of a ternary complex (between polymerase, primed nucleic acid and cognate nucleotide) and detecting the ternary complex using a sensor set forth herein. The methods can include an examination phase followed by a nucleotide incorporation phase.

The examination phase of a detection method can be carried out at a sensor that is positioned to detect association and dissociation of a ternary complex, the ternary complex containing a polymerase, primed template nucleic acid and next correct nucleotide. Ternary complex can be formed by delivering to the sensor one or more components that are capable of forming ternary complex. In some configurations, one or more of the components for forming ternary complex can be immobilized at or near the sensor. As such, the other component(s) can be delivered to the surface, for example, via solution phase delivery, in order to promote ternary complex formation. The solution phase can include one or more of a primed template nucleic acid, polymerase and at least one nucleotide type.

Interaction of polymerase, nucleotide and primed template nucleic acid molecule(s) can be observed at a sensor under conditions where the nucleotide is not covalently added to the primer(s); and the next base in each template nucleic acid can be identified using the observed interaction. The interaction between the primed template, polymerase and nucleotide can be detected in a variety of schemes, for example, as diagrammed in FIGS. 1 through 7. For example, nucleotides that participate in formation of ternary complexes can contain a detectable label. Each nucleotide can have a distinguishable label with respect to other nucleotides. Alternatively, some or all of the different nucleotide types can have the same label and the nucleotide types can be distinguished based on separate deliveries of different nucleotide types to the sensor. In some embodiments, the polymerase can be labeled. Polymerases that are associated with different nucleotide types can have unique labels that distinguish the type of nucleotide to which they are associated. Alternatively, polymerases can have similar labels and the different nucleotide types can be distinguished based on separate deliveries of different nucleotide types to the flow cell.

During the examination phase, discrimination between correct and incorrect nucleotides can be facilitated by ternary complex stabilization. A variety of conditions and reagents can be useful for stabilizing a ternary complex. For example, the primer can contain a reversible blocking moiety that prevents covalent attachment of nucleotide; and/or cofactors that are required for extension, such as divalent metal ions, can be absent; and/or inhibitory divalent cations that inhibit polymerase-based primer extension can be present; and/or the polymerase that is present in the examination phase can have a chemical modification and/or mutation that inhibits primer extension; and/or the nucleotides can have chemical modifications that inhibit incorporation, such as 5′ modifications that remove or alter the native triphosphate moiety.

The type of base that is present at each position along a template can be determined based on observation of the type of nucleotide that participates in formation of a stabilized ternary complex and knowledge of the rules for Watson-Crick base pairing. Generally, an examination step is carried out such that a polymerase and primed template nucleic acid are contacted with at least two different types of nucleotide, each nucleotide being a cognate for a different base suspected of being present in the template. In some configurations, the different nucleotide types are attached to different labels, the labels being distinguishable from each other using a sensor set forth herein. As such, the characteristics of signals collected from a sensor can be used to identity of the next correct nucleotide. Multiple different nucleotide types that are cognates for different base types suspected of being in a template can be present simultaneously during an examination step. Alternatively, a series of nucleotides can be discretely delivered to a primed template nucleic acid, in the presence of polymerase, to form a stabilized ternary complex. The type of nucleotide that forms the complex can be determined from different labels on the nucleotide and/or from knowledge of which nucleotide(s) was(were) present when ternary complex was formed.

Following examination, an extension phase can be carried out by creating conditions where a nucleotide can be added to the primer on each template nucleic acid molecule. Generally, extension occurs in the presence of the sensor; however, the extension step need not be detected since the nucleotide has been identified in a previous examination step. Of course, a method of the present disclosure can include steps for examining ternary complex formation and for detecting the nucleotide that is added to a primer by extension. Detection of primer extension products can be achieved using Sequencing By Synthesis techniques such as those set forth in US Pat. App. Pub. Nos. 2017/0240962 A1; 2018/0051316 A1; 2018/0112265 A1; 2018/0155773 A1 or 2018/0305727 A1; or U.S. Pat. Nos. 9,164,053; 9,829,456; each of which is incorporated herein by reference. The combined information can be used to make base calls at a particular position with higher confidence than using information from only one of detecting ternary complex and detecting extension products.

In some embodiments, extension involves removal of reagents used in the examination phase and replacing them with reagents that facilitate extension. For example, a polymerase used for examination can be replaced with a polymerase that is capable of extension. Similarly, a nucleotide that is used for examination can be replaced with a nucleotide used for extension. By way of more specific example, a native nucleotide or labeled nucleotide that is used to form a stabilized ternary complex can be removed and replaced with a blocked nucleotide that is used for extension. Alternatively, one or more reagents can be added to the examination phase reaction to create extension conditions. For example, catalytic divalent cations can be added to an examination mixture that was deficient in the cations, and/or polymerase inhibitors can be removed or disabled, and/or extension competent nucleotides can be added, and/or a deblocking reagent can be added to convert reversibly blocked primer(s) into extendable primer(s), and/or extension competent polymerase can be added.

Steps for the above nucleic acid detection methods can be carried out cyclically. For example, examination and extension steps of an SBB™ method can be repeated such that in each cycle a single next correct nucleotide is examined (i.e. the next correct nucleotide being a nucleotide that correctly binds to the nucleotide in a template nucleic acid that is located immediately 5′ of the base in the template that is hybridized to the 3′-end of the hybridized primer) and, subsequently, a single next correct nucleotide is added to the primer. Any number of cycles of a sequencing method set forth herein can be carried out including, for example, at least 1, 2, 5, 10, 20, 25, 30, 40, 50, 75, 100, 150 or more cycles. Alternatively or additionally, no more than 150, 100, 75, 50, 40, 30, 25, 20, 10, 5, 2 or 1 cycles are carried out.

Although embodiments of the present disclosure are exemplified herein with regard to sequencing reactions that employ repeated cycles, the cycles need not be repeated nor do the cycles need to include primer extension steps. For example, genotyping can be carried out by examining a single nucleotide position in a template nucleic acid via formation of a stabilized ternary complex. Genotyping can be carried out using serial delivery and/or accumulation of nucleotide cognates for different base types. Examples of genotyping techniques that can be modified to employ the nucleotide delivery methods set forth herein include those set forth in commonly owned U.S. Pat. No. 9,932,631, which is incorporated herein by reference.

Nucleic acid template(s), to be sequenced, can be delivered to a sensor using any of a variety of methods. In some embodiments, a single nucleic acid molecule is to be sequenced at the sensor. The nucleic acid molecule can be delivered to the sensor and can optionally be attached to a solid support surface on or near the sensor. In some configurations, the nucleic acid is subjected to single molecule detection or single molecule sequencing. Alternatively, multiple copies of the nucleic acid can be made and the resulting ensemble can be sequenced. For example, the nucleic acid can be amplified on a surface (e.g. on the conducting channel of a sensor or on a surface that is in a field produced by the channel) using techniques set forth in further detail below.

In multiplex embodiments, a variety of different nucleic acid molecules (i.e. a population having a variety of different sequences) are sequenced. The molecules can optionally be attached to an array of sensors. The nucleic acids can be attached at unique sites of the array and single nucleic acid molecules that are spatially distinguishable one from the other can be sequenced in parallel. Alternatively, the nucleic acids can be amplified on the array to produce a plurality of surface attached ensembles. The ensembles can be spatially distinguishable and sequenced in parallel.

A method set forth herein can use any of a variety of amplification techniques. Exemplary techniques that can be used include, but are not limited to, polymerase chain reaction (PCR), rolling circle amplification (RCA), multiple displacement amplification (MDA), bridge amplification, or random prime amplification (RPA). In particular embodiments, one or more primers used for amplification can be attached to a sensor or to a solid support surface that is proximal to the sensor. In such embodiments, extension of the surface-attached primers along template nucleic acids will result in copies of the templates being attached to the surface. Methods that result in one or more sites on a solid support, where each site is attached to multiple copies of a particular nucleic acid template, can be referred to as “clustering” methods.

In PCR embodiments, one or both primers used for amplification can be attached at or near a sensor. Formats that utilize two species of attached primer are often referred to as bridge amplification because double stranded amplicons form a bridge-like structure between the two attached primers that flank the template sequence that has been copied. Exemplary reagents and conditions that can be used for bridge amplification are described, for example, in U.S. Pat. Nos. 5,641,658 or 7,115,400; U.S. Patent Pub. Nos. 2002/0055100 A1, 2004/0096853 A1, 2004/0002090 A1, 2007/0128624 A1 or 2008/0009420 A1, each of which is incorporated herein by reference. PCR amplification can also be carried out with one of the amplification primers attached to the surface and the second primer in solution. An exemplary format that uses a combination of one solid phase primer and a solution phase primer is known as primer walking and can be carried out to attach amplicon nucleic acids to beads as described in U.S. Pat. No. 9,476,080, which is incorporated herein by reference. Another example is emulsion PCR which can be carried out as described, for example, in Dressman et al., Proc. Natl. Acad. Sci. USA 100:8817-8822 (2003), WO 05/010145, or U.S. Patent Pub. Nos. 2005/0130173 A1 or 2005/0064460 A1, each of which is incorporated herein by reference. The beads produced by emulsion PCR can be deposited in proximity to a sensor set forth herein.

RCA techniques can be used in a method set forth herein. Exemplary reagents that can be used in an RCA reaction and principles by which RCA produces amplicons are described, for example, in Lizardi et al., Nat. Genet. 19:225-232 (1998) or US Pat. App. Pub. No. 2007/0099208 A1, each of which is incorporated herein by reference. Primers used for RCA can be in solution or attached to a solid support surface such as a surface of a sensor.

MDA techniques can also be used in a method of the present disclosure. Some reagents and useful conditions for MDA are described, for example, in Dean et al., Proc Natl. Acad. Sci. USA 99:5261-66 (2002); Lage et al., Genome Research 13:294-307 (2003); Walker et al., Molecular Methods for Virus Detection, Academic Press, Inc., 1995; Walker et al., Nucl. Acids Res. 20:1691-96 (1992); or U.S. Pat. Nos. 5,455,166; 5,130,238; or 6,214,587, each of which is incorporated herein by reference. Primers used for MDA can be in solution or attached to a solid support surface on or near a sensor.

In particular embodiments, a combination of the above-exemplified amplification techniques can be used. For example, RCA and MDA can be used in a combination wherein RCA is used to generate a concatemeric amplicon in solution (e.g. using solution-phase primers). The amplicon can then be used as a template for MDA using primers that are attached to a solid support surface on or near a sensor. In this example, amplicons produced after the combined RCA and MDA steps will be attached on or near the sensor. The amplicons will generally contain concatemeric repeats of a target nucleotide sequence.

A step for detecting ternary complexes can take place during a wash step carried out using a ternary complex stabilizing fluid. The stabilizing fluid can contain Li⁺, betaine or an inhibitory metal cation (e.g. Ca²⁺). Optionally, the stabilizing fluid is held static (i.e., not moving or flowing) during the detection step. However, the fluid can flow, for example, through a flow cell that contains a ternary complex that is to be detected. Exemplary stabilizing fluids and methods for using the stabilizing fluids while detecting ternary complexes are set forth in further detail below or in U.S. 62/662,888, which is incorporated herein by reference.

Examination and detection of a stabilized ternary complex may be accomplished in different ways. For example, monitoring can include measuring association kinetics for the interaction between two or more of the components of the complex. Monitoring the interaction can include measuring equilibrium binding signals or equilibrium binding constants. Thus, for example, the monitoring may include measuring equilibrium binding signals, or the equilibrium binding constant using an electronic sensor. Monitoring the interaction can include, for example, measuring dissociation kinetics of the nucleotide from the primed template nucleic acid in the presence of any one of the four nucleotides. Optionally, monitoring the interaction of the polymerase with the primed template nucleic acid molecule in the presence of a nucleotide molecule includes measuring the kinetics of the dissociation of the closed complex. Techniques for measuring association, equilibrium and dissociation kinetics are known and can be readily modified for use in a method set forth herein by one in the art. See, for example, Markiewicz et al., Nucleic Acids Research 40(16):7975-84 (2012); Xia et al., J. Am. Chem. Soc. 135(1):193-202 (2013); Brown et al., J. Nucleic Acids, Article ID 871939, 11 pages (2010); Washington, et al., Mol. Cell. Biol. 24(2):936-43 (2004); Walsh and Beuning, J. Nucleic Acids, Article ID 530963, 17 pages (2012); and Roettger, et al., Biochemistry 47(37):9718-9727 (2008), which are incorporated by reference herein. It will be understood that a detection technique can accumulate signal over a relatively brief duration as is typically understood to be a single timepoint acquisition. Alternatively, signal can be continuously monitored over time as is typical of a time-based acquisition. It is also possible to acquire a series of timepoints in a periodic fashion to obtain a time-based acquisition.

Some configurations of the methods set forth herein utilize two or more distinguishable signals to distinguish stabilized ternary complexes from each other and/or to distinguish one base type in a template nucleic acid from another base type. For example, two or more charge labels can be distinguished from each other based on unique properties such as unique polarity (e.g. positive vs. negative charge) or unique charge intensity (e.g. one positive charge vs. two positive charges). Intensity differences can result from use of different charge labels and/or different linkers that position labels differently relative to a sensor. Alternatively, the same label type can be used but can be present in different amounts. For example, all members of a first population of ternary complexes can be labeled with a particular charge label, whereas a second population has only half of its members labeled with the charge label. In this example, the second population would be expected to produce half the signal of the first population.

In some embodiments, the examination step is carried out in a way that the identity of at least one nucleotide type is imputed, for example, as set forth in commonly owned U.S. Pat. No. 9,951,385 or US Pat. App. Pub. No. 2018/0305749 A1, each of which is incorporated herein by reference. Accordingly, the present disclosure provides a method of nucleic acid detection that includes the steps of: (a) contacting a primed template nucleic acid with a polymerase and a first mixture of nucleotides under conditions for stabilizing a ternary complex at a nucleotide position in the template, wherein the first mixture includes a nucleotide cognate of a first base type and a nucleotide cognate of a second base type; (b) contacting the primed template nucleic acid with a polymerase and a second mixture of nucleotides under conditions for stabilizing a ternary complex at the nucleotide position in the template, wherein the second mixture includes a nucleotide cognate of the first base type and a nucleotide cognate of a third base type; (c) examining products of steps (a) and (b) for electronic signals produced by a ternary complex that comprises the primed template nucleic acid, a polymerase and a next correct nucleotide, wherein signals acquired for the product of step (a) are ambiguous for the first and second base type, and wherein signals acquired for the product of step (b) are ambiguous for the first and third base type; and (d) disambiguating signals acquired in step (c) to identify a base type that binds the next correct nucleotide.

Alternatively or additionally to using imputation, an examination step can use a decoding or disambiguation scheme to identify one or more nucleotide types, for example, as set forth in commonly owned U.S. Pat. No. 9,951,385 or US Pat. App. Pub. No. 2018/0305749 A1, each of which is incorporated herein by reference. A method of nucleic acid detection can include steps of: (a) sequentially contacting a primed template nucleic acid with first and second mixtures under ternary complex stabilizing conditions, wherein each of the mixtures includes a polymerase and nucleotide cognates for at least two of four different base types in the primed template nucleic acid, wherein the mixtures differ by at least one type of nucleotide cognate; (b) examining the first and second mixtures, or products thereof, separately to detect ternary complexes via an electronic sensor; and (c) identifying the next correct nucleotide for the primed template nucleic acid molecule, wherein the next correct nucleotide is identified as a cognate of one of the four different base types if ternary complex is detected in the two mixtures.

Particular embodiments of the methods set forth herein utilize an encoding scheme that provides for error detection and error correction. Serial examinations of a particular position in the template produce a series of signal states, respectively. For example, different types of ternary complexes can be labeled with different charge labels and the series of signals emitted from the series of examinations can encode the type of nucleotide that is present at the position of the template nucleic acid where the series of ternary complexes formed. Each different nucleotide type is encoded by a unique series of signal states. For sake of explanation, the code can be represented as a series of digits that form a codeword of length n, wherein each digit represents a signal state (e.g. a positive charge or negative charge in the case of a binary digit based on charge polarity) and the length of the codeword is the same as the number of examinations. Error detection is possible when the number of possible codewords exceeds the number of expected nucleotide types. More specifically, error detection is provided since a base call can be identified as valid when it is derived from a codeword that is expected for one of the nucleotide types or invalid when it is derived from a codeword that is not assigned to any nucleotide type.

Moreover, error correction can be provided by an appropriate selection of code complexity and distance between codes for valid base calls. For example, the codewords for each valid base call can differ from the codewords for all other valid base calls by at least three digits. As a consequence, up to two errors per codeword can be detected while a single error can be corrected. Any of a variety of error detecting or error correcting codes used in telecommunications, information theory or coding theory can be adapted for use in a method set forth herein, including but not limited to, a repetition code, parity code, error detecting code, error correcting code, linear code or Hamming code. Exemplary techniques that utilize error detecting codes and that can be adapted for use with a sensor of the present disclosure are set forth in US Pat. App. Pub. No. 2018/0305749 A1, which is incorporated herein by reference.

Accordingly, the present disclosure provides a method of determining a nucleic acid sequence, that includes steps of: (a) contacting a primed template nucleic acid with a series of mixtures for forming ternary complexes, wherein each of the mixtures includes a polymerase and nucleotide cognates for at least two different base types suspected of being present at the next template position of the template nucleic acid; (b) monitoring the next template position for ternary complexes formed by the series of mixtures, wherein a signal state indicates presence or absence of ternary complex formed at the next template position by each individual mixture, thereby determining a series of signal states that encodes a base call for the next template position; and (c) decoding the series of signal states to distinguish a correct base call for the next template position from an error in the base call.

Stabilizing Ternary Complexes

A method of this disclosure can include one or more steps for forming and detecting a ternary complex. Embodiments of the methods exploit the specificity with which a polymerase can form a stabilized ternary complex with a primed template nucleic acid and a next correct nucleotide. The next correct nucleotide can be non-covalently bound to the stabilized ternary complex, interacting with the other members of the complex solely via non-covalent interactions. Useful methods and compositions for forming a stabilized ternary complex are set forth in further detail below and in commonly owned U.S. Pat. App. Pub. Nos. 2017/0022553 A1, 2018/0044727 A1, 2018/0187245 A1 or 2018/0208983 A1, each of which is incorporated herein by reference. Typically, formation and detection of ternary complex is separated from a step of extending the primer, for example, due to reagent exchange between the steps. However, in some embodiments the binding, detection and extension steps can occur in the same mixture.

While a ternary complex can form between a polymerase, primed template nucleic acid and next correct nucleotide in the absence of certain catalytic metal ions (e.g., Mg²⁺), chemical addition of the nucleotide is inhibited in the absence of the catalytic metal ions. Low or deficient levels of catalytic metal ions causes non-covalent sequestration of the next correct nucleotide in a stabilized ternary complex. Other methods disclosed herein also can be used to produce a stabilized ternary complex.

Optionally, a stabilized ternary complex can be formed when the primer of the primed template nucleic acid includes a blocking moiety (e.g. a reversible terminator moiety) that precludes enzymatic incorporation of an incoming nucleotide into the primer. The interaction can take place in the presence of stabilizers, whereby the polymerase-nucleic acid interaction is stabilized in the presence of the next correct nucleotide. The primer of the primed template nucleic acid can optionally be either an extendible primer, or a primer blocked from extension at its 3′-end (e.g., blocking can be achieved by the presence of a reversible terminator moiety on the 3′-end of the primer). The primed template nucleic acid, the polymerase and the cognate nucleotide are capable of forming a stabilized ternary complex when the base of the cognate nucleotide is complementary to the next base of the primed template nucleic acid.

As set forth above, conditions that favor or stabilize a ternary complex can be provided by the presence of a blocking group that precludes enzymatic incorporation of an incoming nucleotide into the primer (e.g. a reversible terminator moiety on the 3′ nucleotide of the primer) or the absence of a catalytic metal ion. Other useful conditions include the presence of a ternary complex stabilizing agent such as an inhibitory metal ion (e.g., a divalent or trivalent inhibitory metal ion) that inhibits polymerase catalyzed nucleotide incorporation or polymerization. Inhibitory metal ions include, but are not limited to, calcium, strontium, scandium, titanium, vanadium, chromium, iron, cobalt, nickel, copper, zinc, gallium, germanium, arsenic, selenium, rhodium, europium, and terbium ions. Optionally, conditions that disfavor or destabilize binary complexes (i.e. complexes between polymerase and primed nucleic acid but lacking cognate nucleotide) are provided by the presence of one or more monovalent cations and/or glutamate anions. As a further example of a stabilizing condition, a polymerase engineered to have reduced catalytic activity or reduced propensity for binary complex formation can be used.

Ternary complex stabilization conditions can accentuate the difference in affinity of polymerase toward primed template nucleic acids in the presence of different nucleotides, for example, by destabilizing binary complexes. Optionally, the conditions cause differential affinity of the polymerase for the primed template nucleic acid in the presence of different nucleotides. By way of example, the conditions include, but are not limited to, high salt and glutamate ions. For example, the salt may dissolve in aqueous solution to yield a monovalent cation, such as a monovalent metal cation (e.g., sodium ion or potassium ion). Optionally, the salt that provides the monovalent cations (e.g., monovalent metal cations) further provides glutamate ions. Optionally, the source of glutamate ions can be potassium glutamate. In some instances, the concentrations of potassium glutamate that can be used to alter polymerase affinity of the primed template nucleic acid extend from 10 mM to 1.6 M of potassium glutamate, or any amount in between 10 mM and 1.6 M.

It will be understood that options set forth herein for stabilizing a ternary complex need not be mutually exclusive and instead can be used in various combinations. For example, a ternary complex can be stabilized by one or a combination of means including, but not limited to, presence of crosslinking of the polymerase domains; crosslinking of the polymerase to the nucleic acid; polymerase mutations that stabilize the ternary complex; allosteric inhibition by small molecules; presence of Li⁺, betaine, uncompetitive inhibitors, competitive inhibitors, or non-competitive inhibitors; absence of catalytic metal ions; presence of a blocking moiety on the primer; or other means set forth herein.

A ternary complex stabilizing fluid can be devoid of one or more components of a ternary complex binding reaction prior to being contacted with a ternary complex. For example, a stabilizing fluid that contains Li⁺, betaine or an inhibitory metal cation (e.g. Ca′) can be devoid of polymerase or nucleotides prior to being contacted with a ternary complex. As such, the stabilizing fluid can function as a wash to remove excess polymerase or nucleotides from a binding reaction that previously functioned to form a ternary complex. In particular embodiments, the concentration of ternary complex in a stabilization fluid is greater than the concentration of free nucleotide and/or polymerase in the fluid. The free nucleotide and polymerase in this fluid can be the same type that is present in the ternary complex. It will be understood that Li⁺, betaine, inhibitory metal cation (e.g. Ca′) or other ternary complex stabilizing agent can be present during ternary complex formation. Alternatively, the ternary complex stabilizing agent can be introduced to a ternary complex that has already been formed.

A particularly useful agent for use in a method or composition of the present disclosure, for example, for stabilizing a ternary complex is lithium. Like the other alkali metals, lithium has a single valence electron that is easily given up to form a cation (Li t). Lithium can be supplied to a reaction in salt form, for example, in the form of LiCl. Lithium, when in contact with ternary complex, can be at a concentration of at least 5 mM, 10 mM, 25 mM, 50 mM, 100 mM, 250 mM or higher. Alternatively or additionally, lithium can be present at a concentration of at most 250 mM, 100 mM, 50 mM, 25 mM, 10 mM, 5 mM or less. Another useful agent for use in a method or composition of the present disclosure, for example, for stabilizing a ternary complex is betaine. Betaine, when in contact with ternary complex, can be at a concentration of at least 1 mM, 10 mM, 50 mM, 100 mM, 500 mM, 1 M, 2 M, 3 M, 3.5M or higher. Alternatively or additionally, betaine can be present at a concentration of at most 3.5 M, 2 M, 1 M, 500 mM, 100 mM, 50 mM, 10 mM, 1 mM, or less. Exemplary conditions and methods that employ lithium and/or betaine for stabilizing ternary complexes is set forth in U.S. Pat. App. Ser. No. 62/662,888, which is incorporated herein by reference.

Inhibitory metal ions can also be used in a method or composition of the present disclosure, for example, as a stabilizing agent. A particularly useful inhibitory metal ion is Ca²⁺. Inhibitory metal ions, when in contact with ternary complex, can be at a concentration of at least 0.1 mM, 0.5 mM, 1 mM, 5 mM, 10 mM, 25 mM, 50 mM, 100 mM, or higher. Alternatively or additionally, inhibitory metal ions can be present at a concentration of at most 100 mM, 50 mM, 25 mM, 10 mM, 5 mM, 1 mM, 0.5 mM, 0.1 mM or less.

A nucleic acid detection method of the present disclosure can involve initial formation of a ternary complex using a plurality of different nucleotide types, and then subsequently examining stability of the complex under a series of changed reagent conditions. These changed conditions can involve progressive removal of one or more nucleotide types from a collection of ternary complexes. For example, a ternary complex that includes a particular nucleotide type will be maintained in a first solution that contains more molecules of that particular nucleotide type. Exchanging the first reagent solution with a second reagent solution that does not include the particular nucleotide type that is present in the ternary complex will cause destabilization of the complex. Detecting the presence of ternary complex in the first solution and dissociation of the ternary complex in the second solution indicates that the identity of the nucleotide type in the ternary complex is the same as the nucleotide type delivered in the first solution. This and other similar ‘washdown’ techniques that can be modified for use on a sensor of the present disclosure are set forth in US Pat. App. Pub. No. 2017/0314064 A1, which is incorporated by reference herein.

Accordingly, a method for identifying the next correct nucleotide for a primed template nucleic acid can include the steps of: (a) providing a stabilized ternary complex including a primed template nucleic acid, polymerase, and next correct nucleotide; (b) contacting the stabilized ternary complex with a mixture that includes a cognate for a first base type suspected of being present in the template; (c) contacting the stabilized ternary complex with a mixture that includes a cognate for a second base type suspected of being present in the template, the second base type being different from the first base type; (d) monitoring the ternary complex in contact with the second mixture to detect any of the stabilized ternary complex remaining after step (b) and step (c); and (e) identifying the nucleotide that comprises the base complementary to the next base of the template strand using results from step (d).

The present disclosure provides a method for sequencing a primed template nucleic acid. The method can include steps of (a) providing a sensor that includes a first electrode, a second electrode, a conduction channel operably connecting the first electrode to the second electrode and a primed template nucleic acid that is immobilized at the sensor; (b) contacting the primed template nucleic acid with a polymerase and the next correct nucleotide, thereby forming a stabilized ternary complex that includes the primed template nucleic acid, the polymerase and the next correct nucleotide, wherein the stabilized ternary complex is prevented from covalently incorporating the next correct nucleotide into the primed template nucleic acid; (c) monitoring the sensor to detect a signal produced by the stabilized ternary complex, whereby the next correct nucleotide is identified from the signal; (d) adding a next correct nucleotide to the primer of the primed template nucleic acid after step (b), thereby producing an extended primer; and (e) repeating steps (a) through (d) for the primed template nucleic acid that includes the extended primer.

In an alternative configuration, a method for sequencing a primed template nucleic acid can include steps of (a) providing a sensor that includes a first electrode, a second electrode, a conduction channel operably connecting the first electrode to the second electrode and a polymerase that is immobilized at the sensor; (b) contacting the polymerase with a primed template nucleic acid and the next correct nucleotide, thereby forming a stabilized ternary complex that includes the primed template nucleic acid, the polymerase and the next correct nucleotide, wherein the stabilized ternary complex is prevented from covalently incorporating the next correct nucleotide into the primed template nucleic acid; (c) monitoring the sensor to detect a signal produced by the stabilized ternary complex, whereby the next correct nucleotide is identified from the signal; (d) adding a next correct nucleotide to the primer of the primed template nucleic acid after step (b), thereby producing an extended primer; and (e) repeating steps (a) through (d) for the primed template nucleic acid that includes the extended primer.

A method for sequencing a primed template nucleic acid can also include steps of (a) providing a sensor having a first electrode that is attached to a polymerase via a first conductive linker and a second electrode that is attached to a primed template nucleic acid via a second conductive linker; (b) contacting the polymerase and the primed template nucleic acid with the next correct nucleotide, thereby forming a stabilized ternary complex including the primed template nucleic acid, the polymerase and the next correct nucleotide, wherein the stabilized ternary complex is prevented from covalently incorporating the next correct nucleotide into the primed template nucleic acid; (c) monitoring the sensor to detect a circuit between the first and second electrode produced by the stabilized ternary complex, whereby the next correct nucleotide is identified from the signal; (d) adding a next correct nucleotide to the primer of the primed template nucleic acid after step (b), thereby producing an extended primer; and (e) repeating steps (a) through (d) for the primed template nucleic acid that includes the extended primer.

In other embodiments, a method for sequencing a primed template nucleic acid can include steps of (a) providing a sensor having a first electrode that is attached to a polymerase via a first conductive linker and a second electrode that is attached to a nucleotide via a second conductive linker; (b) contacting the polymerase and the nucleotide with a primed template nucleic acid, thereby forming a stabilized ternary complex including the primed template nucleic acid, the polymerase and the nucleotide, wherein the stabilized ternary complex is prevented from covalently incorporating the nucleotide into the primed template nucleic acid; and (c) monitoring the sensor to detect a circuit between the first and second electrode produced by the stabilized ternary complex, whereby the nucleotide is identified as the next correct nucleotide for the primed template nucleic acid; (d) adding a next correct nucleotide to the primer of the primed template nucleic acid after step (b), thereby producing an extended primer; and (e) repeating steps (a) through (d) for the primed template nucleic acid that includes the extended primer.

Alternatively, a method for sequencing a primed template nucleic acid can include steps of (a) providing a sensor having a first electrode that is attached to a primed template nucleic acid via a first conductive linker and a second electrode that is attached to a nucleotide via a second conductive linker; (b) contacting the primed template nucleic acid and the nucleotide with a polymerase, thereby forming a stabilized ternary complex including the primed template nucleic acid, the polymerase and the nucleotide, wherein the stabilized ternary complex is prevented from covalently incorporating the nucleotide into the primed template nucleic acid; and (c) monitoring the sensor to detect a circuit between the first and second electrode produced by the stabilized ternary complex, whereby the nucleotide is identified as the next correct nucleotide for the primed template nucleic acid; (d) adding a next correct nucleotide to the primer of the primed template nucleic acid after step (b), thereby producing an extended primer; and (e) repeating steps (a) through (d) for the primed template nucleic acid that includes the extended primer.

Throughout this application various publications, patents and/or patent applications have been referenced. The disclosures of these documents in their entireties are hereby incorporated by reference in this application.

A number of embodiments have been described. Nevertheless, it will be understood that various modifications may be made. Accordingly, other embodiments are within the scope of the following claims.

1. A method for identifying the next correct nucleotide for a primed template nucleic acid, comprising
(a) providing a sensor comprising a first electrode, a second electrode, a conduction channel operably connecting the first electrode to the second electrode and a primed template nucleic acid that is immobilized at the sensor;
(b) contacting the primed template nucleic acid with a polymerase and the next correct nucleotide, thereby forming a stabilized ternary complex comprising the primed template nucleic acid, the polymerase and the next correct nucleotide, wherein the stabilized ternary complex is prevented from covalently incorporating the next correct nucleotide into the primed template nucleic acid; and
(c) monitoring the sensor to detect a signal produced by the stabilized ternary complex, whereby the next correct nucleotide is identified from the signal.

2. The method of claim 1, wherein the primed template nucleic acid is immobilized at the sensor by a linker that directly attaches the primed template nucleic acid to the conduction channel.

3. The method of claim 2, wherein the linker is configured to conduct voltage or current between the primed template nucleic acid and the conduction channel.

4. The method of claim 2, wherein the linker is insulated from conducting voltage or current between the primed template nucleic acid and the conduction channel.

5. The method of claim 2, wherein the linker is covalently attached to the primer of the primed template nucleic acid.

6. The method of claim 2, wherein the linker is covalently attached to the template of the primed template nucleic acid.

7. The method of claim 1, wherein the conduction channel comprises the primed template nucleic acid, whereby the primed template nucleic acid operably connects the first electrode to the second electrode.

8. The method of claim 7, wherein a first non-nucleic acid linker connects the primed template nucleic acid to the first electrode

9. The method of claim 8, wherein a second non-nucleic acid linker connects the primed template nucleic acid to the second electrode.

10. The method of claim 7, wherein the primed template nucleic acid is directly attached to the first electrode or the second electrode.

11. The method of claim 1, wherein the primed template nucleic acid is immobilized at the sensor by a linker that is insulated from conducting voltage or current between the primed template nucleic acid and the sensor.

12. The method of claim 1, wherein the primed template nucleic acid is immobilized in an electric field produced by the sensor.

13. The method of claim 1, wherein the signal is produced by a charge label that is attached to the next correct nucleotide by a linker.

14. The method of claim 13, wherein the charge label is attached to the next correct nucleotide by an uninterrupted chain of covalent bonds.

15. The method of claim 13, wherein the charge label is attached to the next correct nucleotide by a receptor-ligand affinity pair other than the polymerase and the primed template nucleic acid.

16. The method of claim 1, wherein the signal is produced by a charge label that is attached to the polymerase by a linker.

17. The method of claim 16, wherein the charge label is attached to the polymerase by an uninterrupted chain of covalent bonds.

18. The method of claim 16, wherein the charge label is attached to the polymerase by a receptor-ligand affinity pair other than the next correct nucleotide and the primed template nucleic acid.

19. The method of claim 1, wherein step (b) further comprises contacting the primed template nucleic acid with a second nucleotide that comprises a different type of base compared to the next correct nucleotide.

20. The method of claim 19, wherein the next correct nucleotide is attached to an exogenous label that produces the signal and the second nucleotide is attached to an exogenous label that produces a second signal that is distinguished from the signal by the sensor.

21.-122. (canceled)
